(19) United States
(12) Patent Application Publication
(10) Pub. No.: US 2017/0035365 A1
(43) Pub. Date: Feb. 9, 2017

(54) BIOLOGICAL INFORMATION PROCESSING SYSTEM, ELECTRONIC APPARATUS, SERVER SYSTEM AND BIOLOGICAL INFORMATION PROCESSING METHOD

(71) Applicant: Seiko Epson Corporation, Tokyo (JP)

(72) Inventors: Minoru Hasegawa, Suwa-shi (JP); Masao Kuroda, Shiojiri-shi (JP); Ichiro Aoshima, Matsumoto-shi (JP)

(21) Appl. No.: 15/106,503

(22) PCT Filed: Jan. 14, 2015
(86) PCT No.: PCT/JP2015/000143

§ 371 Date: Jun. 20, 2016

(30) Foreign Application Priority Data
Jan. 15, 2014 (JP) 2014-005151
Mar. 13, 2014 (JP) 2014-049769

(51) Int. Cl. A61B 5/00; A61B 5/16; A61B 5/0205

(52) U.S. Cl. CPC A61B 5/7285; A61B 5/681; A61B 5/0205; A61B 5/165; A61B 5/4812; A61B 5/4866; A61B 5/6898; A61B 5/7225; A61B 5/743; A61B 5/0002; A61B 5/02405

(57) ABSTRACT

A biological information processing system includes a heart rate information acquisition unit that acquires heart rate information of a user, a determination unit that determines a basal heart rate based on the heart rate information, and an update unit that determines an update condition of the basal heart rate and performs update processing of the basal heart rate if a determination that the update condition is satisfied is made.

### TECHNICAL FIELD

This application contains the contents of Japanese Patent Application 2014-005151 filed on Jan. 15, 2014 and Japanese Patent Application 2014-049769 filed on Mar. 13, 2014.

The present invention relates to a biological information processing system, an electronic apparatus, a server system, a biological information processing method, etc.

### BACKGROUND ART

In related art, apparatuses and systems that acquire heart rate information of users and provide information on health, etc. of users based on the acquired information have been used. The heart rate information may be acquired based on sensor information acquired from pulse sensor and heartbeat sensor, for example. Here, the heart rate refers to the number of beats of a heart and the pulse rate refers to the number of beats of arteries after transmission of pressure generated by blood pushed out from the heart by the beating of the heart to peripheral vessels. Normally, the heart rate and the pulse rate are equal in an able-bodied person.

The heart rate information itself (e.g., heart rate value) may be used as an index value indicating the health condition of the user, and, by given calculation using the heart rate information, information on the daily lifestyle habit of the user may be obtained. For example, a technique of calculating calorie consumption of the user based on the heart rate information and presenting the information to the user or the like is known.

For determination of the health condition of the user, not only using the heart rate information acquired by measurements but also performing comparison processing between the heart rate information and the heart rate as reference is considered. Generally, the heart rate values significantly differ among individual users, and, in the condition determination of the users, appropriate determination can be made using relative values with respect to the reference value or the like, not the absolute value of the heart rate.

For example, PTL 1 discloses a determination technique using “minimum pulse rate” as the reference. Further, PTL 2 discloses a technique of calculating calorie consumption of a user based on the heart rate information and presenting it to the user.

Furthermore, PTL 3 discloses a technique of grasping the health condition of a person to be measured using basal resting pulses. In addition, PTL 4 discloses a technique of acquiring biological information while suppressing the effect of noise using a basic pulse frequency and a resting pulse frequency.

Note that “basal resting pulse rate” in PTL 3 refers to a pulse rate when a user (a person to be measured, a person wearing an apparatus) is in an awake state and a rest state, and has a concept close to “resting heart rate” in the embodiment. Similarly, “resting pulse rate” in PTL 4 has a concept close to “resting heart rate” in the embodiment. Further, “basic pulse rate” is expected to be a lower value than the resting pulse rate, but is information in the awake state, and is not completely equal to “basal heart rate” in the embodiment.

### CITATION LIST

### Patent Literature

### PTL 1: JP-A-4-180730

### PTL 2: JP-A-2009-285498

### PTL 3: JP-A-2012-157435

### PTL 4: JP-A-2011-212384

### SUMMARY OF INVENTION

### Solution to Problem

An aspect of the invention relates to a biological information processing system including a heart rate information acquisition unit that acquires heart rate information of a user, a determination unit that determines a basal heart rate based on the heart rate information, and an update unit that determines an update condition of the basal heart rate and performs update processing of the basal heart rate if a determination that the update condition is satisfied is made.

Another aspect of the invention relates to a biological information processing system including a heart rate information acquisition unit that acquires heart rate information of a user, and a processing unit that obtains a resting heart rate as the heart rate information determined to correspond to a rest state of the user based on the heart rate information, and calculates a basal heart rate based on the resting heart rate.

Still another aspect of the invention relates to a biological information processing system including a heart rate information acquisition unit that acquires heart rate information of a user, and a processing unit that obtains a basal heart rate as the heart rate information determined to correspond to a deep sleep state of the user based on the heart rate information, and calculates a resting heart rate based on the obtained basal heart rate.

Yet another aspect of the invention relates to an electronic apparatus including the biological information processing system.

Still yet another aspect of the invention relates to a server system including the biological information processing system.

Further another aspect of the invention relates to a biological information processing method including acquiring heart rate information of a user, determining a basal heart rate based on the heart rate information, and determining an update condition of the basal heart rate and performing update processing of the basal heart rate if a determination that the update condition is satisfied is made.

Still further another aspect of the invention relates to a biological information processing method including acquiring heart rate information of a user, and obtaining a resting heart rate as the heart rate information determined to correspond to a rest state of the user based on the heart rate information, and calculating a basal heart rate based on the resting heart rate.

Yet further another aspect of the invention relates to a biological information processing method including acquiring heart rate information of a user, and obtaining a basal heart rate as the heart rate information determined to correspond to a deep sleep state of the user based on the heart rate information, and calculating a resting heart rate based on the obtained basal heart rate.

### BRIEF DESCRIPTION OF DRAWINGS

The invention will be described with reference to the accompanying drawings, wherein like numbers reference like elements.

FIG. 1 shows a configuration example of a biological information processing system according to embodiments.

FIG. 2 shows a detailed configuration example of the biological information processing system according to the embodiment.

FIG. 3 shows a configuration example of a wearable apparatus including the biological information processing system.

FIG. 4A shows an example of an electronic apparatus according to the embodiment.

FIG. 4B shows an example of an electronic apparatus according to the embodiment.

FIG. 5 shows a configuration example of a portable terminal apparatus including the biological information processing system.

FIG. 6 shows a cooperation example of the wearable apparatus and the portable terminal apparatus.

FIG. 7 is a basic flowchart showing update processing of a basal heart rate.

FIG. 8 is a flowchart showing measurement processing performed at intervals of several seconds.

FIG. 9 is an explanatory diagram of a technique of obtaining the minimum heart rate from heart rate histograms.

FIG. 10 is another explanatory diagram of the technique of obtaining the minimum heart rate from heart rate histograms.

FIG. 11 is a diagram for explanation of update processing of the basal heart rate using the minimum heart rate in related art.

FIG. 12 is a diagram for explanation of processing of increasing the basal heart rate.

FIG. 13 shows a relationship diagram between the age and the basal heart rate.

FIG. 14 shows an example of a table showing relationships between the age and the basal heart rate.

FIG. 15 is a detailed flowchart for explanation of update processing of the basal heart rate.

FIG. 16 shows a comparison example between a technique of the embodiment and a technique of related art.

FIG. 17 shows another comparison example between the technique of the embodiment and the technique of related art.

FIG. 18A is an explanatory diagram of factors at body motion and non-body motion.

FIG. 18B is an explanatory diagram of factors at body motion and non-body motion.

FIG. 19 shows correlations between basal metabolic rates calculated by the technique of related art and the technique of the embodiment.

FIG. 20 shows an example of a home screen displayed on a display unit.

FIG. 21 shows an example of a factor setting screen displayed on the display unit.

FIG. 22 shows an example of a heart rate trend screen displayed on the display unit.

FIG. 23A shows a screen example intuitively presenting health degree information.

FIG. 23B shows a screen example intuitively presenting health degree information.

FIG. 24 shows an example of an analysis screen displayed on the display unit.

FIG. 25 shows an example when an initial value of the basal heart rate is set to a higher value with respect to a user.

FIG. 26 shows an example when the initial value of the basal heart rate is set to a lower value with respect to the user.

FIG. 27 shows a configuration example of a biological information processing system according to the embodiment.

FIG. 28 is a flowchart for explanation of processing of the second embodiment.

FIG. 29 is a flowchart for explanation of processing of classifying heart rate information based on body motion information.

FIG. 30A shows actual measurement data of heart rate information.

FIG. 30B shows examples of respective histograms created based on heart rate information.

FIG. 31 is a flowchart for explanation of processing of the third embodiment.

FIG. 32 shows actual measurement data of a user having smaller differences in heart rate information between a sleep state and an awake state.

FIG. 33A shows actual measurement data of heart rate information.

FIG. 33B shows examples of respective histograms created based on heart rate information.

FIG. 34 is a flowchart for explanation of processing of a modified example.

### DESCRIPTION OF EMBODIMENTS

According to some aspects of the invention, a biological information processing system, an electronic apparatus, a server system, a biological information processing method, etc. that accurately perform processing using heart rate information by obtaining a basal heart rate from the heart rate information and performing update processing with respect to the basal heart rate based on a given update condition may be provided.

Further, according to some aspects of the invention, a biological information processing system, an electronic apparatus, a server system, a biological information processing method, etc. that appropriately acquire a basal heart rate and a resting heart rate regardless of a lifestyle or the like of a user by performing calculation processing one from the other in consideration of a relationship between the basal heart rate and the resting heart rate may be provided.

One embodiment of the invention relates to a biological information processing system including a heart rate information acquisition unit that acquires heart rate information of a user, a determination unit that determines a basal heart rate based on the heart rate information, and an update unit that determines an update condition of the basal heart rate and performs update processing of the basal heart rate if a determination that the update condition is satisfied is made.

In the embodiment, the basal heart rate is determined from the heart rate information and the basal heart rate is updated based on the determination of the update condition. Thereby, the basal heart rate may be used as reference or the like for processing using the heart rate information and the basal heart rate is appropriately updated, and thereby, it may be possible to improve processing accuracy or the like.

Further, in the embodiment, the update unit may increase the basal heart rate if the update condition is not satisfied in a predetermined period or at a predetermined number of times.

Thereby, it may be possible to perform the processing of increasing the basal heart rate.

Furthermore, in the embodiment, the update unit may count a number of times of repetition from a start of measurement to an end of the measurement of the heart rate information as the predetermined number of times.

Thereby, it may be possible to perform counting of the predetermined number of times in units of the start and the end of measurement or the like.

In addition, in the embodiment, a body motion information acquisition unit that acquires body motion information from a body motion sensor may be further provided, and the update unit may count a number of times when body motion of the user is determined to be smaller of the number of times of repetition based on the body motion information in a period from the start of the measurement to the end of the measurement as the predetermined number of times.

Thereby, it may be possible to perform counting of the predetermined number of times using the start and the end of the measurement and the body motion information or the like.

Further, in the embodiment, the update unit may perform determination of the update condition of the basal heart rate based on a minimum heart rate obtained from the heart rate information.

Thereby, it may be possible to perform the determination of the update condition based on the minimum heart rate.

Furthermore, in the embodiment, the update unit may obtain the minimum heart rate by performing moving average processing on the heart rate information acquired in a given minimum heart rate measurement period.

Thereby, it may be possible to accurately obtain the minimum heart rate or the like.

In addition, in the embodiment, the update unit may obtain a histogram representing a relationship between heart rate values and frequencies at which the respective heart rate values are detected based on the heart rate information acquired in the minimum heart rate measurement period, and, in a range of the heart rate value from x to x+n (x, n are given positive numbers), obtain x for which the frequency exceeds a given frequency threshold value and the value is minimum as the minimum heart rate.

Thereby, it may be possible to accurately obtain the minimum heart rate or the like.

Further, in the embodiment, the determination unit may perform processing of replacing a default value of the basal heart rate set by the biological information processing system by the minimum heart rate obtained from the heart rate information, and the update unit may perform the update processing of updating the basal heart rate determined by the determination unit.

Thereby, it may be possible to perform processing with respect to the default value and perform the update processing with respect to the subsequent basal heart rate.

Furthermore, in the embodiment, a body motion information acquisition unit that acquires body motion information from a body motion sensor may be further provided, and, if a determination that a period in which body motion of the user is determined to be smaller is contained in a given minimum heart rate measurement period is made based on the body motion information in the minimum heart rate measurement period, the determination unit may employ the minimum heart rate obtained based on the heart rate information in the minimum heart rate measurement period as the basal heart rate.

Thereby, in the processing with respect to the default value, it may be possible to use the body motion information or the like.

In addition, in the embodiment, a body motion information acquisition unit that acquires body motion information from a body motion sensor may be further provided, and the update unit may determine the update condition of the basal heart rate based on the body motion information.

Thereby, in the update processing, it may be possible to use the body motion information or the like.

Further, in the embodiment, the predetermined number of times may be a number of times when a determination of the update condition of the basal heart rate is made.

Thereby, it may be possible to use the number of times of determination of the update condition as the predetermined number of times.

Furthermore, in the embodiment, the update unit may increase the basal heart rate if a predetermined period lapses.

Thereby, it may be possible to increase the basal heart rate on condition of the lapse of the predetermined period.

In addition, in the embodiment, a health degree information calculation unit that obtains relative information between the basal heart rate and the heart rate information, and obtains health degree information representing a health degree based on the relative information may be further provided.

Thereby, it may be possible to obtain the health degree information from the basal heart rate and the heart rate information.

Another embodiment of the invention relates to a biological information processing system including a heart rate information acquisition unit that acquires heart rate information of a user, and a processing unit that obtains a resting heart rate as the heart rate information determined to correspond to a rest state of the user based on the heart rate information, and calculates a basal heart rate based on the resting heart rate.

In the embodiment, the basal heart rate is calculated based on the resting heart rate. Accordingly, even in the case where it is impossible to directly measure the basal heart rate depending on the lifestyle or the like of the user, it may be possible to appropriately set the basal heart rate using the actually measured heart rate information or the like.

Further, in one aspect of the invention, a body motion information acquisition unit that acquires body motion information of the user may be further provided, and the processing unit may calculate a first heart rate based on the resting heart rate obtained from the heart rate information having the body motion information at a signal level equal to or more than a given threshold value, obtain a second heart rate from the heart rate information having the body motion information at the signal level less than the given threshold value, and determine the basal heart rate by comparison processing between the first heart rate and the second heart rate.

Thereby, it may be possible to obtain the first and second heart rates in response to the levels of the body motion information and determine the basal heart rate from the comparison processing of them or the like.

Furthermore, in the embodiment, the processing unit may employ smaller one of the first heart rate and the second heart rate as the basal heart rate.

Thereby, it may be possible to determine the basal heart rate from comparison processing of the magnitude relationship between the first and second heart rates or the like.

In addition, in the embodiment, the processing unit may calculate the first heart rate by performing calculation processing on the resting heart rate using a given calculation formula.

Thereby, it may be possible to calculate the first heart rate from the resting heart rate using the given calculation formula or the like.

Further, in the embodiment, a body motion information acquisition unit that acquires body motion information of the user may be further provided, and the processing unit may determine whether the acquired heart rate information is information in a sleep state or information in an awake state based on the body motion information, and calculate the basal heart rate based on a determination result.

Thereby, it may be possible to determine the sleep state or the awake state and calculate the basal heart rate or the like.

Furthermore, in the embodiment, the processing unit may obtain a minimum heart rate obtained from the heart rate information determined to be the information in the awake state as the resting heart rate, and calculate the basal heart rate based on the obtained resting heart rate.

Thereby, it may be possible to obtain the resting heart rate from the heart rate information corresponding to the awake state and calculate the basal heart rate from resting heart rate or the like.

In addition, in the embodiment, in the case where awake heart rate information as the heart rate information determined to be the information in the awake state and sleep heart rate information as the heart rate information determined to be the information in the sleep state are acquired and a difference or ratio between the awake heart rate information and the sleep heart rate information is equal to or less than a given threshold value, the processing unit may obtain a minimum heart rate in a measurement period as the resting heart rate.

Thereby, even in the case where the changes in heart rate information are smaller between the awake state and the sleep state, it may be possible to appropriately obtain the resting heart rate or the like.

Further, in the embodiment, if the heart rate information is determined to be information in the sleep state, the processing unit may employ a minimum heart rate obtained from the heart rate information as the basal heart rate.

Thereby, it may be possible to obtain the basal heart rate based on the heart rate information corresponding to the sleep state or the like.

Furthermore, in the embodiment, the processing unit may calculate the basal heart rate by performing calculation processing on the resting heart rate using a given calculation formula.

Thereby, it may be possible to calculate the basal heart rate from the resting heart rate using the given calculation formula or the like.

In addition, in the embodiment, if a measurement period of the heart rate information is equal to or more than a predetermined period, the processing unit may perform calculation of the basal heart rate based on the resting heart rate.

Thereby, it may be possible to determine whether or not to perform calculation of the basal heart rate based on the length of the measurement period or the like.

Another aspect of the invention relates to a biological information processing system including a heart rate information acquisition unit that acquires heart rate information of a user, and a processing unit that obtains a basal heart rate as the heart rate information determined to correspond to a deep sleep state of the user based on the heart rate information, and calculates a resting heart rate based on the obtained basal heart rate.

In the embodiment, the resting heart rate is calculated based on the basal heart rate. Accordingly, even in the case where it is impossible to directly measure the resting heart rate depending on the lifestyle or the like of the user, it may be possible to appropriately set the resting heart rate using the actually measured heart rate information or the like.

Further, in the embodiment, the processing unit may calculate calorie consumption of the user using the resting heart rate.

Thereby, it may be possible to calculate the calorie consumption of the user using the resting heart rate or the like.

Furthermore, in the embodiment, a body motion information acquisition unit that acquires body motion information of the user may be further provided, and the processing unit may determine whether the acquired heart rate information is information in a sleep state or information in an awake state based on the body motion information, and calculate the resting heart rate based on a determination result.

Thereby, it may be possible to determine the sleep state or the awake state and calculate the resting heart rate or the like.

In addition, in the embodiment, the processing unit may calculate the resting heart rate by performing calculation processing on the basal heart rate using a given calculation formula.

Thereby, it may be possible to calculate the resting heart rate from the basal heart rate using the given calculation formula or the like.

Another aspect of the invention relates to an electronic apparatus including the biological information processing system.

Another aspect of the invention relates to a server system including the biological information processing system.

Another aspect of the invention relates to a biological information processing method including acquiring heart rate information of a user, determining a basal heart rate based on the heart rate information, and determining an update condition of the basal heart rate and performing update processing of the basal heart rate if a determination that the update condition is satisfied is made.

Another aspect of the invention relates to a biological information processing method including acquiring heart rate information of a user, and obtaining a resting heart rate as the heart rate information determined to correspond to a rest state of the user based on the heart rate information, and calculating a basal heart rate based on the resting heart rate.

Another aspect of the invention relates to a biological information processing method including acquiring heart rate information of a user, and obtaining a basal heart rate as the heart rate information determined to correspond to a deep sleep state of the user based on the heart rate information, and calculating a resting heart rate based on the obtained basal heart rate.

As below, embodiments of the invention will be explained. The embodiments to be explained are not unduly limit the contents of the invention described in the appended claims. Further, not all of the configurations explained in the embodiments are necessarily essential component elements of the invention.

### First Embodiment

### 1.1. Outline of First Embodiment

Various techniques of determining conditions (a health condition, etc.) of users from heart rate information have been known. For example, in PTL 2, a technique of actually measuring heart rate information (HR) using a heart rate sensor, etc. and obtaining calorie consumption from minute oxygen consumption (VO₂) estimated based on the heart rate information is known. When calorie intake exceeds the calorie consumption, it may be possible to determine that the user is suspected of exacerbation of metabolic syndrome or the like, and the calorie consumption may be used as health degree information representing the health condition of the user.

In PTL 2, when the minute oxygen consumption is estimated from the heart rate information, the following formula (1), etc. are used.

$$\begin{matrix}[ Math . 1 ] & \\ \frac{( {VO}_{2} - {VO}_{2 r} )}{( {VO}_{2 m} - {VO}_{2 r} )} \times 100 ( \% ) - \frac{( HR - {HR}_{r} )}{( {HR}_{m} - {HR}_{r} )} \times 100 ( \% ) & ( 1 )\end{matrix}$$

VO₂ₘ in the formula (1) is the maximum value of the minute oxygen consumption. VO₂ᵣ is minute oxygen consumption in a rest state, HRₘ is the maximum value of the heart rate information, and HRᵣ is a value of the heart rate information in the rest state. In PTL 1, the respective values of VO₂ₘ, VO₂ᵣ, HRₘ, HRᵣ are obtained and VO₂ is obtained from those values and the actually measured HR. VO₂ and the calorie consumption have a given relationship, and it may be possible to obtain the calorie consumption from the estimated VO₂.

VO₂ₘ is the maximum value of the minute oxygen consumption, however, it is not practical to make a subject to do exercise with high load to the degree at which the minute oxygen consumption is considered to be the maximum. Accordingly, it may be impossible to obtain VO₂ₘ from the actual measurement values, and a given statistical value (hypothetical value) is used. Similarly, it may be impossible to obtain HRₘ from the actual measurement values, and a given statistical value is used. On this account, individual differences among users are not considered for VO₂ₘ and HRₘ. Therefore, with respect to a certain number of people, when a tendency of calorie consumption of a group or the like is obtained, the formula (1) is effective, however, when the calorie consumption of every person is obtained, using the formula (1) remains problematic.

Further, HRᵣ as the value of the heart rate information at rest is used for the estimation of VO₂ based on the formula (1), however, HRᵣ is also problematic. As activities of a human with energy consumption, not only physical activities (exercise) but mental activities are considered. Further, in the mental activities, the value of heart rate information rises and the calorie consumption also increases. That is, even when the user is physically at rest, the value of HRᵣ differs between the case without mental activities (e.g., a sleep state) and the case with mental activities (e.g., the case of complex thinking such as calculation or tension).

In PTL 1, variations of HRᵣ in mental activities are not considered. Accordingly, when the user is exercising with relatively high load, the formula (1) is effective, however, when the user is not exercising or the like, correct calculation of calorie consumption may be impossible. In related art, the calculation of calorie consumption is assumed to be performed during exercise and the problem due to the formula (1) is not significant. For example, in related art, it is only necessary to inform the user of energy consumed by exercise when the user does the exercise of running or the like, and importance is not given to measurement of calorie consumption at rest. However, in the health degree determination in daily activities of the user, the calorie consumption at rest is an important index. For example, in the above described determination of exacerbation of metabolic syndrome, it is necessary to perform comparison between the calorie intake and the calorie consumption in units of given periods (e.g., continuous 24 hours) including resting times. In addition, when various kinds of health degree information is considered, the necessity of correct calculation of the calorie consumption by the mental activities at rest is high, and the technique using the formula (1) having a problem of accuracy at rest is not appropriate.

Therefore, the applicant introduces a technique using a basal heart rate for more accurate processing using the heart rate information. As an example, the basal heart rate (HR₀) is obtained and the calorie consumption or the like is calculated using the basal heart rate. In the following explanation, an example of obtaining health degree information representing the health condition of the user such as calorie consumption will be explained, and using the basal heart rate for other processing is not hindered.

Here, the basal heart rate represents the heart rate information when the user is in a deep sleep state. In the deep sleep state, variations due to mental activities are not caused unlike the above described HR and it may be possible to accurately perform processing using the heart rate information not only at exercise (body motion) but also at rest (non-body motion).

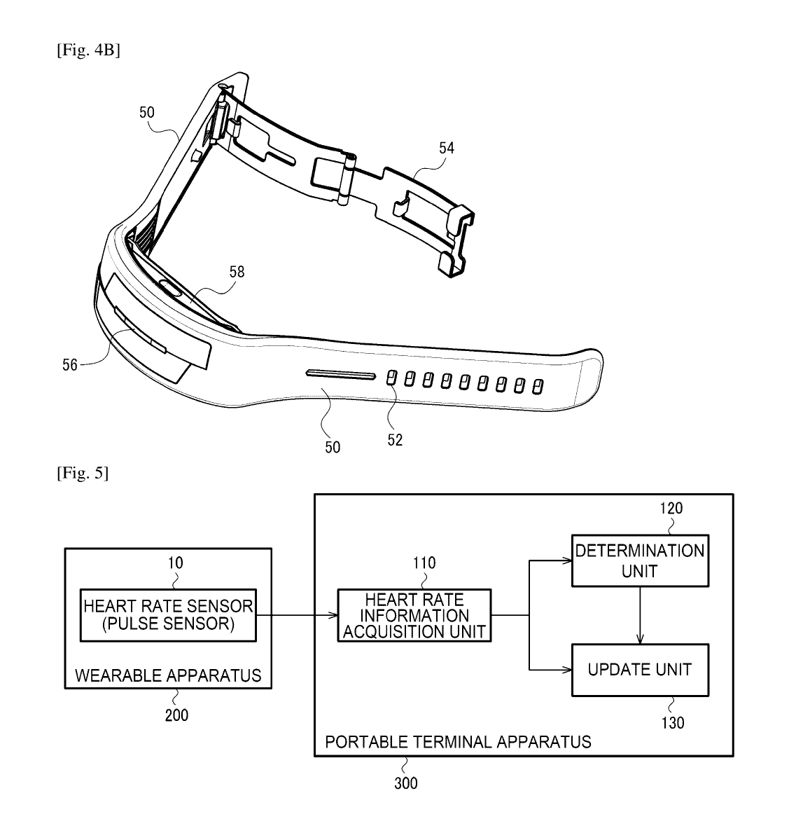

Note that the basal heart rate in the embodiment is not necessarily heart rate information in the deep sleep state. For example, the time in the deep sleep state in daily sleep is about several hours and the time differs among individual users. That is, for some user, the period in the deep sleep state is very short or does not exist depending on the day. Further, in consideration of the tendency of the time in the deep sleep state decreasing with advancing age or the like, a situation that it is impossible to measure the heart rate in the deep sleep state even using an apparatus according the embodiment (e.g., a wearable apparatus, which will be described later using FIG. 4A, etc.) is conceivable. In the first place, it is difficult to determine whether or not the user is in the deep sleep state without using information of brain wave or the like, and, with the apparatus according to the embodiment alone, it is difficult to ensure that the obtained heart rate is a real heart rate in the deep sleep state.

Therefore, the basal heart rate according to the embodiment is not necessarily limited to that representing the heart rate in the deep sleep state, but may be information determined as heart rate information in the deep sleep state by determination processing in the biological information processing system of the embodiment. That is, for example, when an algorithm for obtaining the basal heart rate with a certain degree of accuracy is packaged in the biological information processing system, the basal heart rate in the embodiment includes the information obtained using the algorithm.

Using the basal heart rate, compared to the case using the above described HRᵣ, etc., it may be possible to accurately perform the processing using the heart rate information. Note that, even when the basal heart rate is used, it is necessary to appropriately perform update processing of the basal heart rate. According to the researches by the applicant, it is known that the basal heart rate varies less from day to day, however, as biological information, a certain degree of variations are caused and it is desirable to update the basal heart rate using the actually measured heart rate information or the like. However, as described above, the basal heart rate is reference of the processing using the heart rate information. When the reference frequently varies, the determination result undesirably varies. Specifically, when the determination result (calorie consumption, stress level, or the like) changes, it may be impossible for the user to clearly distinguish whether the change is caused by variations in life, physical condition, or the like of the user or the change is caused by variations in basal heart rate as reference.

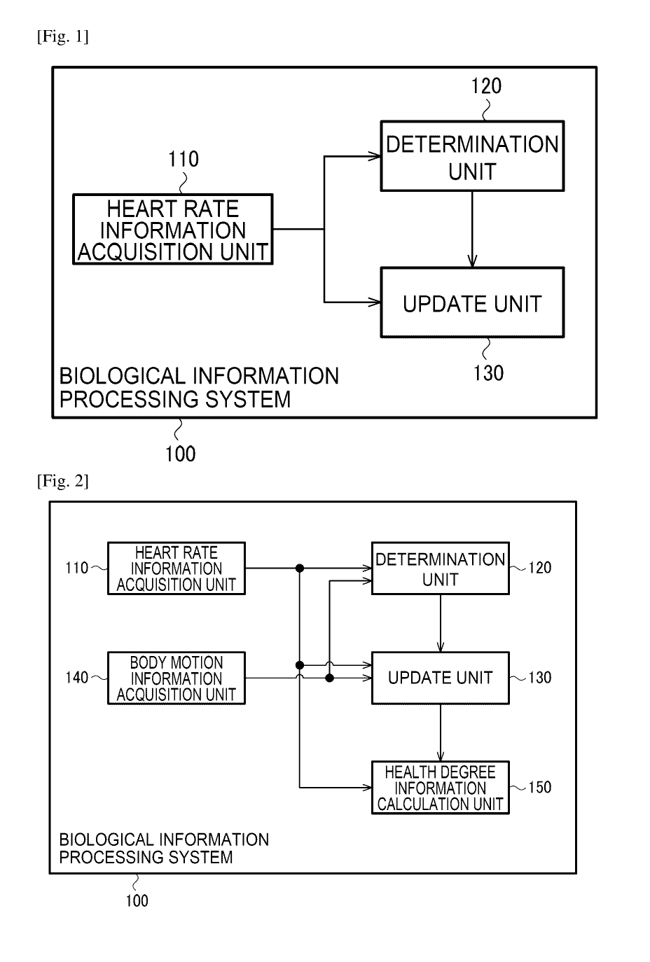

Accordingly, the applicant proposes a technique using the basal heart rate and updating the basal heart rate based on an appropriate update condition. Specifically, as shown in FIG. 1, a biological information processing system 100 according to the embodiment includes a heart rate information acquisition unit 110 that acquires heart rate information of a user, a determination unit 120 that determines a basal heart rate based on the heart rate information, and an update unit 130 that performs determination of an update condition of the basal heart rate and update processing of the basal heart rate if a determination that the update condition is satisfied is made.

In this manner, the basal heart rate may be used and the basal heart rate may be appropriately updated, and it may be possible to accurately perform the processing using the heart rate information. The details of the update condition of the basal heart rate, etc. will be described later.

As below, a system configuration example of the biological information processing system 100 according to the embodiment or the like will be explained, then, the details of the update processing of the basal heart rate will be explained. Here, a way of obtaining a default value of the basal heart rate (a value independent of the actual measurement value of the heart rate information) or the like will be explained. Finally, processing of obtaining health degree information will be explained as a specific example of processing using the heart rate information and the basal heart rate. In the case of using the basal heart rate, it may be possible to obtain other health degree information than the calorie consumption. Specifically, whether or not the user is in the deep sleep state may be determined using the heart rate information and the basal heart rate, and deep sleep time information representing the time in the deep sleep state may be obtained from the determination. Or, whether or not stress is applied to the user may be determined using the heart rate information and the basal heart rate, and stress information representing the time of stress application or the like may be obtained from the determination.

### 1.2. System Configuration Example

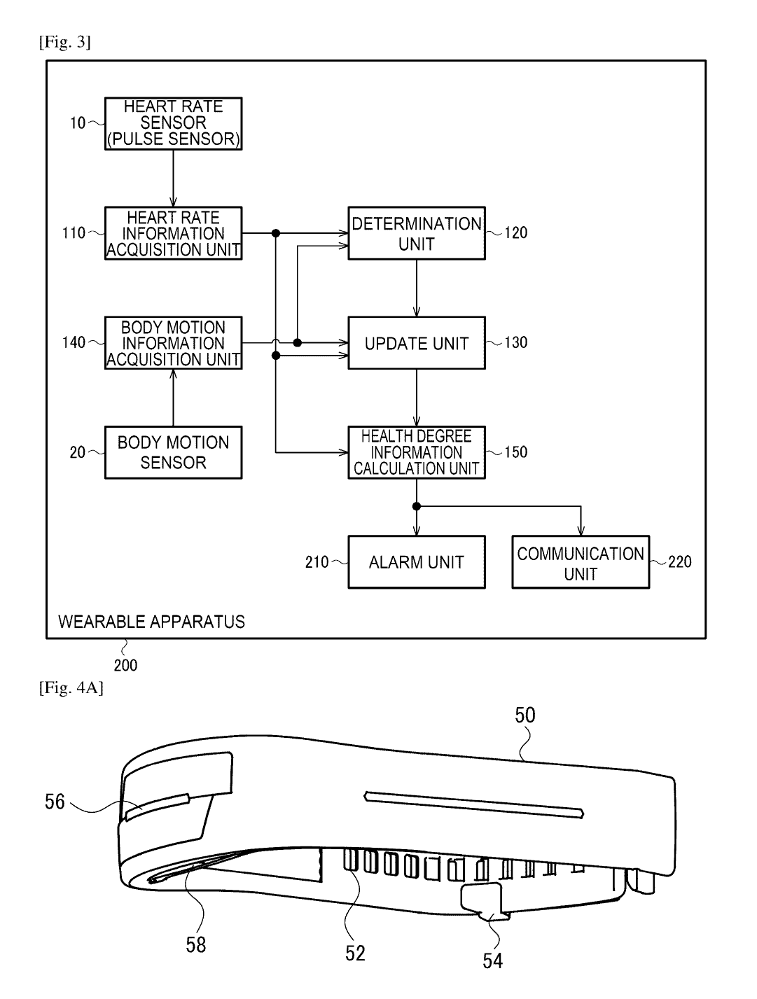

FIG. 2 shows a detailed configuration example of the biological information processing system 100 according to the embodiment. As shown in FIG. 2, the biological information processing system 100 includes the heart rate information acquisition unit 110, the determination unit 120, the update unit 130, a body motion information acquisition unit 140, and a health degree information calculation unit 150. Note that the biological information processing system 100 is not limited to the configuration in FIG. 2, but various modifications by omitting part of these component elements or adding other component elements may be made. Further, various modifications may be similarly made in FIG. 3, etc.

The heart rate information acquisition unit 110, the determination unit 120, and the update unit 130 are the same as those in FIG. 1 and their detailed explanation will be omitted. The body motion information acquisition unit 140 acquires body motion information from a body motion sensor. Here, the body motion sensor is a sensor that detects motion of the user as an acquisition object of the heart rate information, may be an acceleration sensor, an angular velocity sensor (gyro sensor), or another sensor, or these sensors may be combined. The body motion information acquired by the body motion information acquisition unit 140 is used for update processing of the basal heart rate or the like. The details will be described later.

The health degree information calculation unit 150 obtains relative information between the basal heart rate and the heart rate information, and obtains health degree information representing the health degree based on the relative information. The health degree information here is information representing the health condition of the user, including e.g., calorie consumption, deep sleep time information, and stress information. The details of the processing in the health degree information calculation unit 150 will be described later.

Further, the technique of the embodiment may be applied to an electronic apparatus including the biological information processing system 100 in FIGS. 1 and 2, etc. The electronic apparatus here may be a biological information measurement apparatus, or, in a restricted sense, a wearable apparatus worn by the user. In this case, the measurement by the heart rate sensor is performed in the wearable apparatus as the electronic apparatus according to the embodiment.

Specifically, as shown in FIG. 3, the electronic apparatus according to the embodiment (wearable apparatus 200) includes a heart rate sensor 10 (pulse sensor, pulse wave sensor), a body motion sensor 20, the heart rate information acquisition unit 110, the determination unit 120, the update unit 130, the body motion information acquisition unit 140, the health degree information calculation unit 150, an alarm unit 210, and a communication unit 220. As shown in Ha. 2, the heart rate information acquisition unit 110, the determination unit 120, the update unit 130, the body motion information acquisition unit 140, the health degree information calculation unit 150, etc. in FIG. 3 correspond to the biological information processing system 100 according to the embodiment.

The heart rate sensor 10 is a sensor that performs measurement of the heart rate. For the body motion sensor 20, various sensors may be used as described above. In the case where the electronic apparatus is the wearable apparatus 200, the heart rate information acquisition unit 110 acquires sensor information from the heart rate sensor 10 within the apparatus (or a result of signal processing for the sensor information) as heart rate information. Similarly, the body motion information acquisition unit 140 acquires sensor information from the body motion sensor 20 within the apparatus (or a result of signal processing for the sensor information) as body motion information.

The alarm unit 210 informs (presents) the health degree information calculated in the health degree information calculation unit 150 to the user. Various alarming forms in the alarm unit 210 are conceivable, and may emit sound or voice, light a light emitting part of an LED, or vibrate a vibrating part. Further, the alarm unit 210 may be realized using a display unit that displays various display screens, and the display unit may be realized using a liquid crystal display, an organic EL display, or the like.

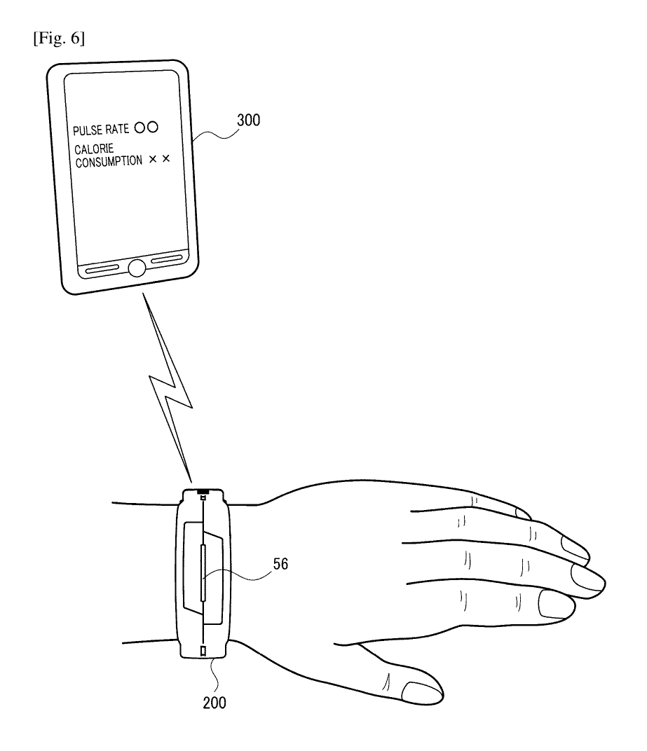

The communication unit 220 performs communication with other apparatuses via various networks. As will be described later using FIG. 6, alarming in other apparatuses of the calculation result in the wearable apparatus 200 is conceivable, and, in this case, the obtained health degree information and information for presenting the health degree information (e.g., information of the display screen or the like) is transmitted to the other apparatuses.

FIGS. 4A and 4B show an example of the wearable apparatus 200 here. As shown in FIGS. 4A and 4B, the wearable apparatus 200 may be a band-shaped (wrist-watch) electronic apparatus including a band 50, band holes 52, and a buckle 54. In the example in FIGS. 4A and 4B, the wearable apparatus includes a light emitting part 56 as the alarm unit 210, and informs the user of various kinds of information including the health degree information by turning on an LED or blinking an LED or the like. Further, the wearable apparatus 200 includes a case 58, and the heart rate sensor 10, the body motion sensor 20 and an electronic substrate, etc. that realize the biological information processing system 100 are housed in the case 58.

Further, the electronic apparatus according to the embodiment is not limited to the wearable apparatus 200 including the heart rate sensor 10, etc., but may be other electronic apparatuses. For example, the electronic apparatus of the embodiment may be a portable terminal apparatus such as a smartphone. In this case, as shown in FIG. 5, the portable terminal apparatus 300 includes the heart rate information acquisition unit 110, the determination unit 120, and the update unit 130 corresponding to the biological information processing system 100, and the heart rate information acquisition unit 110 acquires heart rate information from the heart rate sensor 10 mounted on the wearable apparatus 200 or the like. In this case, the wearable apparatus 200 and the portable terminal apparatus 300 are connected via various networks including short-range wireless communication. Note that, though not illustrated in FIG. 5, the portable terminal apparatus 300 may include the alarm unit and the communication unit like the wearable apparatus 200 in FIG. 3.

FIG. 6 shows a specific example. In FIG. 6, the band-shaped wearable apparatus 200 shown in FIG. 4A, etc. and the portable terminal apparatus 300 such as a smartphone are connected via short-range wireless communication or the like, and information of the heart rate, etc. calculated based on the information from the heart rate sensor 10 mounted on the wearable apparatus 200 is displayed on the display unit of the portable terminal apparatus 300. Note that, in FIG. 6, the alarm unit 210 (light emitting part 56) is also provided in the wearable apparatus 200, and thereby, the processing result using the heart rate information may be informed by the wearable apparatus 200. In this case, in the configuration of FIG. 5, the processing result is acquired in the portable terminal apparatus 300 through the procedure that, first, the wearable apparatus 200 transmits the sensor information to the portable terminal apparatus 300, and the portable terminal apparatus 300 calculates the health degree information using the sensor information and transmits the calculation result to the wearable apparatus 200.

Note that, as described above, FIG. 6 corresponds to the configuration in FIG. 3, and, in this case, also the processing result using the heart rate information is acquired in the wearable apparatus 200 and communication or the like is not particularly unnecessary with respect to the alarming in the light emitting part 56. In this case, the calculation of the health degree information or the like is not performed in the portable terminal apparatus 300, and, when display in the display unit of the portable terminal apparatus 300 or the like is performed, it is necessary to receive the calculation result from the wearable apparatus 200.

Further, the electronic apparatus according to the embodiment is not limited to the wearable apparatus 200 or the portable terminal apparatus 300, but various apparatuses including PC (Personal Computer) may be used.

The technique of the embodiment may be applied to a server system including the biological information processing system 100. A configuration example of the server system is the same as the portable terminal apparatus 300 in FIG. 5. Note that, in this case, it is conceivable that the server system is provided in a location physically far from the user and, if the processing result in the server system is informed by the alarm unit of the server system, it is impossible for the user to recognize it. Therefore, in the server system, it is preferable to transmit the calculation result of the health degree information or the like to the apparatus such as the wearable apparatus 200 or the portable terminal apparatus 300 used by the user.

Generally, the server system has higher processing performance and a less limited storage area of a storage unit than the wearable apparatus 200 and the portable terminal apparatus 300. Therefore, compared to the case where the wearable apparatus 200 or the like contains the biological information processing system 100, it may be possible to perform faster processing using heart rate information or the like. If the storage area is larger, when log data of the heart rate information or the like is acquired, it may be possible to store log data of many users and increase the volume of log data per person. Accordingly, highly versatile processing is performed using many pieces of user data and the heart rate information of users for several or several tens of years, and thereby, improvement in calculation accuracy of the health degree information with respect to the users, etc. can be expected.

Various communication routes between the wearable apparatus 200 and the server system are conceivable. For example, in the case where the wearable apparatus 200 is directly connectable to a network, the wearable apparatus 200 may directly communicate with the server system via the network. Alternatively, communication may be made between the wearable apparatus 200 and the server system via another apparatus such that, first, the wearable apparatus 200 may transmit sensor information to the portable terminal apparatus 300 using short-range wireless communication or the like and the portable terminal apparatus 300 may transfer the sensor information to the server system via the network.

### 1.3 Update Processing of Basal Heart Rate

Next, the details of update processing of basal heart rate will be explained. Specifically, first, calculation processing of the minimum heart rate and update processing of the basal heart rate using the minimum heart rate will be explained. This corresponds to update processing of decreasing the basal heart rate. Then, update processing of increasing the basal heart rate will be explained. Further, setting and updating of the default value of the basal heart rate and processing using body motion information will be explained, and finally, the whole update processing of the basal heart rate will be explained using a flowchart, etc.

1.3.1 Calculation of Minimum Heart Rate

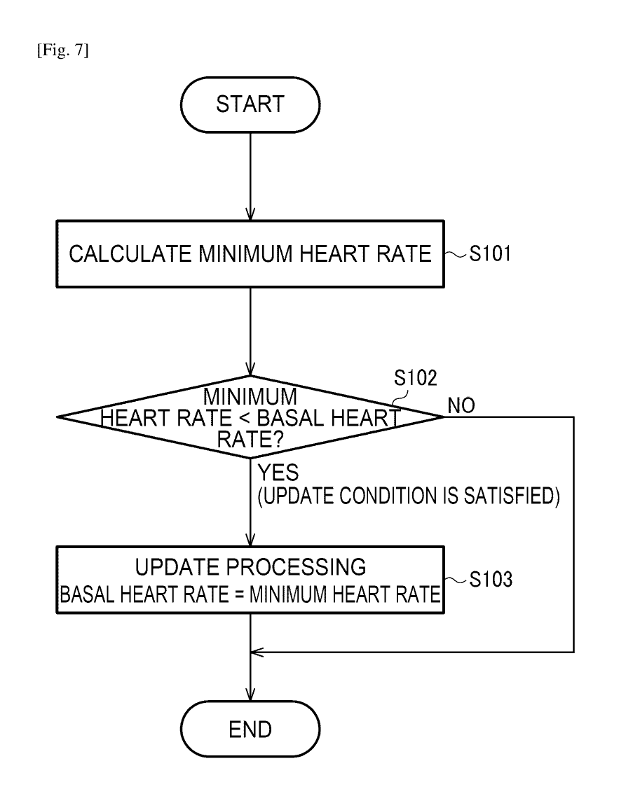

In the embodiment, the update unit 130 obtains the minimum heart rate based on the heart rate information and determines an update condition of the basal heart rate based on the minimum heart rate. As described above, the basal heart rate represents the heart rate in the deep sleep state or the heart rate determined to be in the deep sleep state. Accordingly, the heart rate assumed to be in the smallest numerical value among various states including the deep sleep state, a light sleep state, the rest state, and the exercise state that can be taken by a subject user is the basal heart rate. That is, if the basal heart rate does not vary, the value is not lower than the basal heart rate at measurement of the heart rate information, and, if the value of the heart rate information is lower than the basal heart rate, the basal heart rate may be updated by the lowered value. Specifically, as shown in FIG. 7, first, calculation of the minimum heart rate is performed (S101), and whether or not the minimum heart rate is lower than the basal heart rate is determined (S102). If Yes at S102, the update condition of the basal heart rate is satisfied and the basal heart rate is updated to the value of the minimum heart rate (S103).

Note that, the reality is that the heart rate may be falsely detected due to body motion artifact or the like depending on the apparatus attachment condition at heart rate measurement, and, if update is performed simply when the value is lower than the lower limit value, there is a risk of false updating. Accordingly, in the embodiment, first, the calculation accuracy of the minimum heart rate at S101 is raised. The minimum heart rate here is a value obtained, with a period from the start of measurement to the end of measurement of the heart rate information as a unit, based on the heart rate information within the period. Further, the period from the start of measurement to the end of measurement is from pressing down of a start button to pressing down of a stop button by the user. In the case of a band-shaped apparatus in FIG. 4A, etc., a modification using a period from when the apparatus is wrapped around the user's arm to when the apparatus is detached or the like may be made.

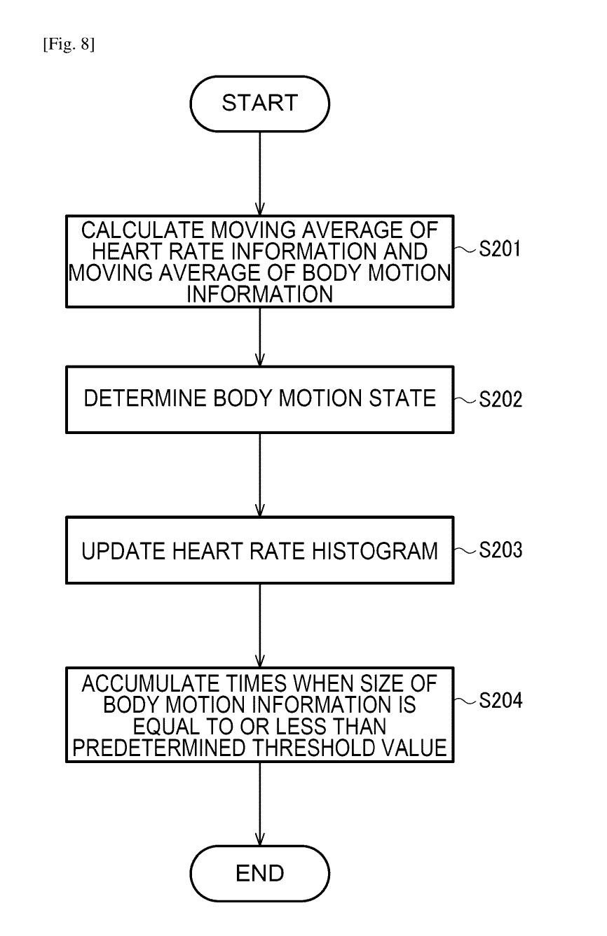

Specifically, the update unit 130 obtains the minimum heart rate by performing moving arrange processing on the heart rate information acquired in a given minimum heart rate measurement period. In the minimum heart rate measurement period, processing shown in a flowchart of FIG. 8 is performed on a regular basis (e.g., one every several seconds).

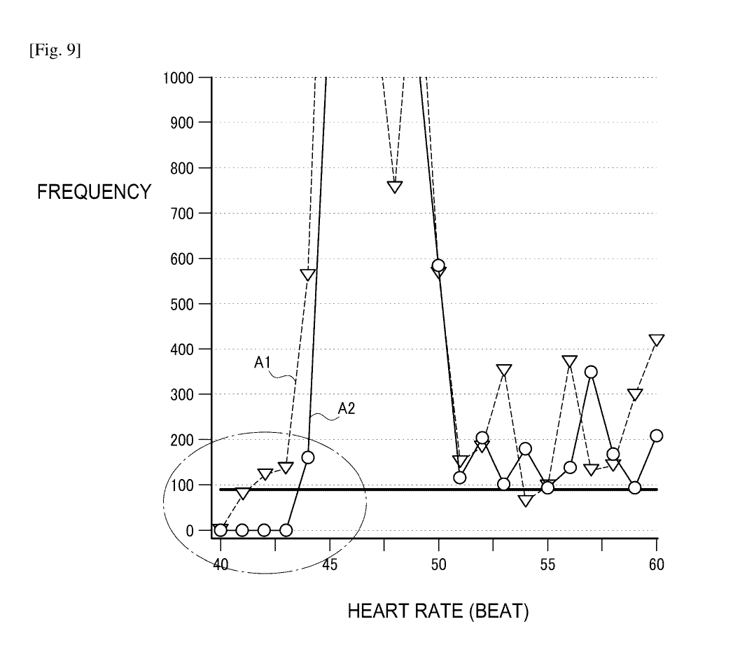

When the processing is started, first, heart rates HR at the moments are acquired from the heart rate sensor 10 and body motion information (e.g., acceleration value acc) at the moments is acquired from the body motion sensor 20. Then, a moving average is taken with respect to HR (and acc) (S201). Regarding HR, if the acquired values are used as they are, many of the values vary and, particularly, significantly vary near the lower limit in histograms in FIG. 9, etc. Further, the minimum heart rate is a value closer to the minimum value as described above, and is obtained using information near the lower limit with significant variations, and thereby, the calculation accuracy is lower. On the other hand, the variations of the values may be suppressed by taking the moving average, and thereby, it may be possible to accurately obtain the minimum heart rate.

FIG. 9 shows a specific example. FIG. 9 shows histograms showing numerical values of heart rates along the horizontal axis and frequencies at which the heart rates appear within the minimum heart rate measurement period along the vertical axis. A1 (plot with inverted triangles) is a histogram before moving average processing and A2 (plot with circles) is a histogram after moving average processing. In FIG. 9, a given frequency threshold value is set so that the very small values estimated to be falsely detected may not be employed as the minimum heart rate and, here, the minimum heart rate with the frequency exceeding 90 is employed as the minimum heart rate. Nevertheless, the value significantly varies on A1 before moving average processing, and the frequency may be larger to some extent in a range in which the numerical values of the heart rate are smaller. In the example of FIG. 9, it is not preferable to employ the heart rate of about 41 to 43 as the minimum heart rate, however, 42 is the minimum heart rate if the threshold determination is performed.

On the other hand, the variations of the value are suppressed near the lower limit as shown by A2 through the moving average processing, and rise of the graph may be made steeper. As a result, the minimum heart rate is 44 and the possibility of employing the excessively smaller value as the minimum heart rate may be prevented.

After the moving average processing at S201, determination of the body motion state is performed (S202). For example, the state here represents whether the body motion is frequent or not in a short-term span and, more specifically, the state is determined using comparison processing between an average value of body motion signals for about one minute (or an average value of difference values) and a threshold value, periodicity of signals, or the like. Then, the histogram is updated using the heart rate after the moving average processing calculated at S201. Specifically, the frequency of the value of the heart rate calculated at S201 may be increased by one.

Further, in the flowchart of FIG. 8, determination as to whether or not the user is sleeping based on the body motion information obtained with the heart rate information at S201 is assumed. Accordingly, at S204, whether or not the body motion signal is smaller than a predetermined value is determined and lengths in the periods in which the signal is smaller than the predetermined value are accumulated and stored. The details of the processing using the body motion signals at S201 and S204 will be described later.

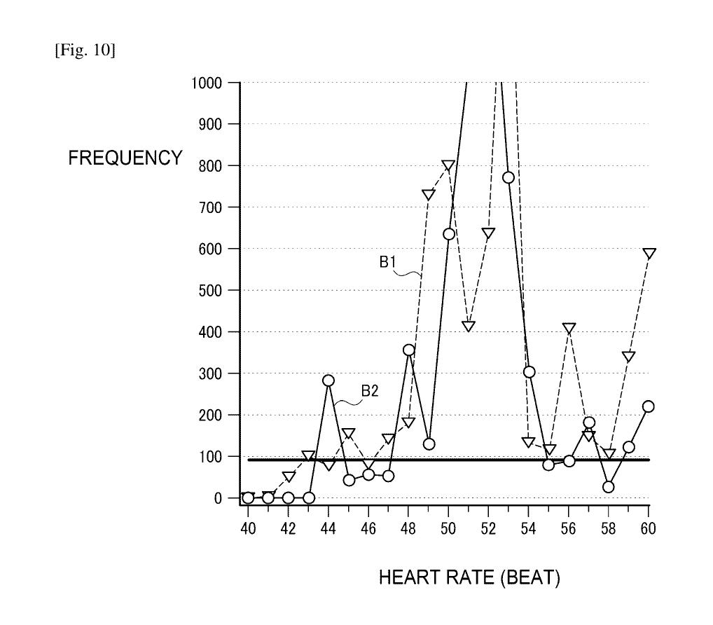

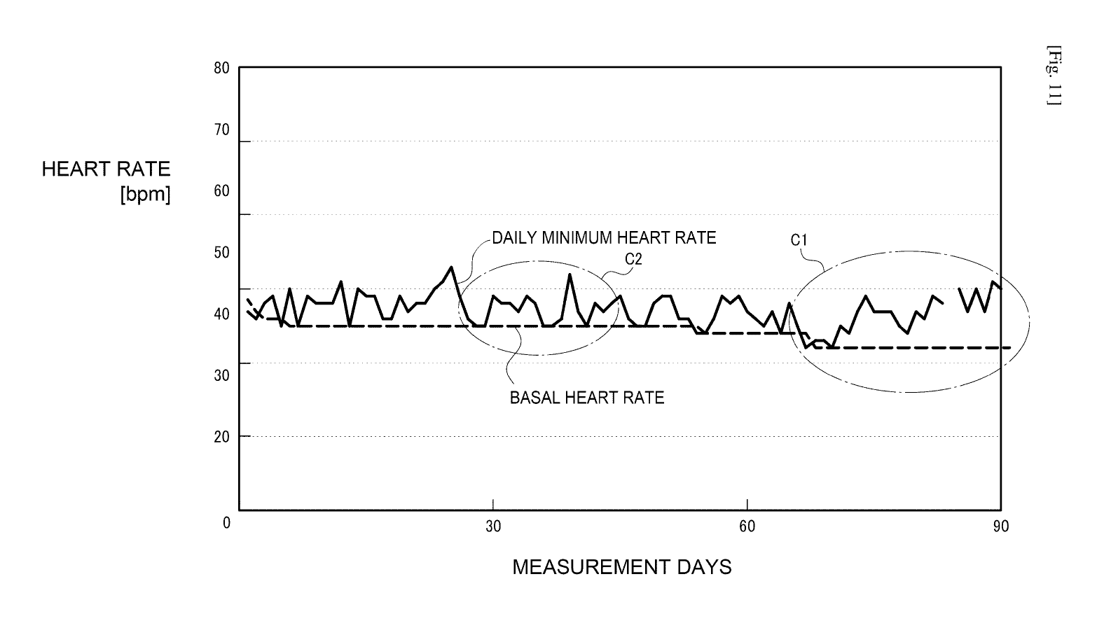

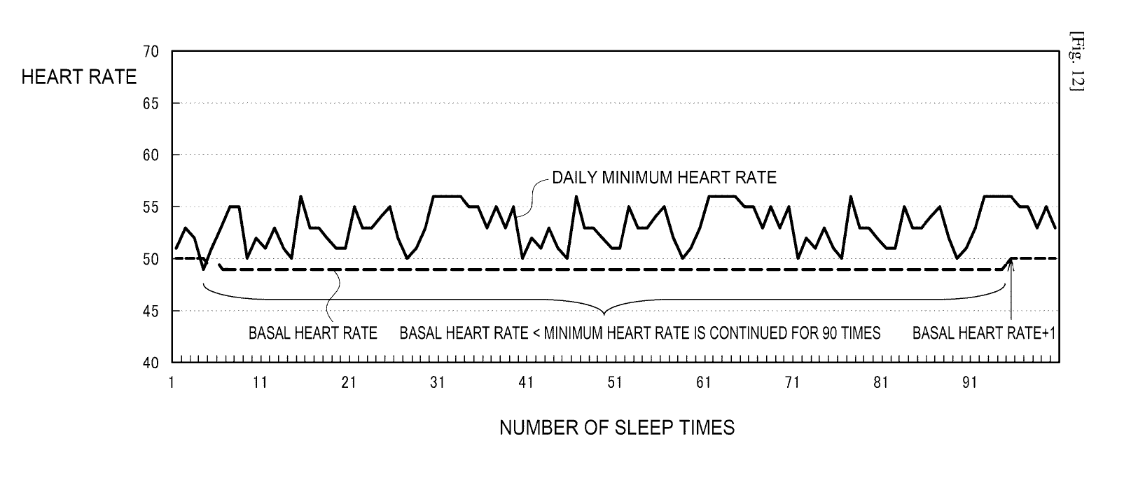

Note that, even when the moving average processing is performed as described above, a singular point may appear in the histogram. FIG. 10 shows a specific example. In FIG. 10, B1 is a histogram before moving average processing and B2 is a histogram after moving average processing. In comparison between B1 and B2, variations may be suppressed by the moving average processing, and the frequency of the heart rate of 42, 43 may be suppressed to be lower in B2 than that in B1. As a result, regarding B1, the minimum heart rate is 43, but 43 may be excluded from the minimum heart rate.

Regarding B2, the frequency is larger at the heart rate of 44, and the minimum heart rate is 44 from threshold determination. However, in consideration of the very small frequencies at 45 to 48, the frequency at 44 is a singular value caused as a result of measurement errors and moving average calculation, and the minimum heart rate of 44 is inappropriate.

Therefore, in the embodiment, the minimum heart rate may be obtained using not a frequency at a given heart rate value but also frequencies at the previous and next heart rate values. Specifically, the update unit 130 obtains a histogram representing a relationship between the heart rate values and the frequencies at which the respective heart rate values are detected based on the heart rate information acquired in the minimum heart rate measurement period, and obtains x having a frequency exceeding a given frequency threshold value and the minimum value in a range from x to x+n (x, n are given positive numbers) of the heart rate values as the minimum heart rate.

In the example of Ha. 10, the horizontal axis indicates integer values and x is a natural number. Further, e.g., “2” may be used as n. In this case, if the frequencies exceed the threshold value at all of the heart rate values x, x+1, x+2, x is employed as the minimum heart rate. In the case of FIG. 10, if x=44, the frequency at 44 exceeds the threshold value, but the frequencies do not exceed the threshold value at x+1=45 and x+2=46. Therefore, it may be possible not to determine x=44 as the minimum heart rate. Then, at x=48, the frequencies exceed the threshold value at all of 48, 49, 50, and three continuous heart rate numerical values exceeding the threshold value are not found if x is less than 48. Therefore, it may be possible to determine the minimum heart rate as 48 and accurately obtain the minimum heart rate.

Note that the technique of obtaining the minimum heart rate is not limited to searching for the minimum heart rate value continuously exceeding the threshold value. For example, searches are made from the larger values, and the numerical value for which the frequency monotonously decreases and the decrease in the monotonous decrease range is the maximum may be specified.

For example, in FIG. 10 (though the upper part of the graph is missing), suppose that the frequency at the heart rate=52 is the maximum. In this case, the monotonous decrease ranges when searches are made from the larger numerical values toward the smaller numerical values (i.e., monotonous increase ranges from the smaller to the larger numerical values) are three ranges from 52 to 49, from 48 to 47, and from 44 to 43. In this case, in comparison among the decrease in the range from 52 to 49, the decrease in the range from 48 to 47, the decrease in the range from 44 to 43, the decrease in the range from 52 to 49 is the maximum. In this case, the lower limit value in the monotonous decrease range, i.e., 49 may be employed as the minimum heart rate.

Ideally, the histograms as shown in FIG. 10 have single peak (monotonous increase in a range smaller than the peak and monotonous decrease in a range larger than the peak). In FIG. 10, some other smaller peaks appear than the original single peak by the singular points, however, it is clear that one having the maximum height (increase, decrease) of the peaks is the originally desired peak. That is, using the above described technique, like the technique of searching for the value continuously exceeding the threshold value, it may be possible to obtain the appropriate minimum heart rate by excluding the singular points.

As is clear from that the minimum heart rate is 48 in the searching for the value continuously exceeding the threshold value and the minimum heart rate is 49 in the searching for the value with larger decrease, the value of the minimum heart rate may change depending on the used technique. Further, which technique is appropriate depends on the shape of the actually acquired histogram or the like. In the case where the measurement period contains both periods in the sleep state and the awake state, the histogram changes depending on the use condition of the user so that two peaks corresponding to the sleep state and the awake state may appear as the peaks of the histogram. That is, how to obtain the minimum heart rate is not limited to the above described techniques, but various modifications may be made such that another technique is used or some techniques are combined.

1.3.2 Processing of Increasing Basal Heart Rate

In the above described manner, it may be possible to accurately obtain the minimum heart rate, and thereby, appropriately update the basal heart rate. However, the above described update processing is to decrease the basal heart rate, and update processing of increasing the rate is not considered.

It is known that the basal heart rate changes due to changes in environments and temperatures. For example, a person usually living in a high-attitude location moves to a low-attitude location, and thereby, oxygen partial pressure increases and normal oxygen intake may be acquired with the smaller heart rate. Accordingly, there are cases where the basal heart rate singularly changes (particularly, decreases). As described above, by only updating simply when the minimum heart rate is below the basal heart rate, after the singular cases, the reference may not match the normal life environment.

FIG. 11 shows a specific example. In FIG. 11, the basal heart rate is shown by a dotted line and the minimum heart rate actually measured by the technique (e.g., the minimum heart rate on a daily basis) is shown by a solid line. In FIG. 11, the minimum heart rates having the smaller values due to environmental changes or the like are acquired after lapses of about 50 days and 70 days, and accordingly, the update processing of decreasing the basal heart rate is performed. Then, after a lapse of about 80 days, the environment returns.

In this case, like before the lapse of 50 days, the heart rate of the subject user returns to the higher level than that during the environmental change, and, as known from the range surrounded by C1 in FIG. 11, the basal heart rate is not updated from the lower level. As a result, though the user lives a normal life, the daily heart rates are larger relative to the basal heart rate. Specifically, the magnitude (ratio, or increased amount) of the heart rate measured in C1 of FIG. 11 relative to the basal heart rate is larger than the magnitude (ratio, or increased amount) of the heart rate measured in C2 relative to the basal heart rate. Accordingly, though the user lives the normal life, determinations of too heavy exercise load, feeling stress, insufficient sleep are made. For example, the solid line is broken in C1 of FIG. 11, and this shows that a false detection of not getting sleep is made though the user actually sleeps.

As disclosed in PTL 1, in the processing using heart rate information, techniques using the lowest value for the subject user or an equivalent value as reference are widely used. Therefore, there have been the techniques of updating the reference value to the smaller value using actual measurement values in related art, however, a technique of updating the reference value to the larger value is unknown.

The applicant performs update processing of increasing the basal heart rate. Specifically, if an update condition is not satisfied in a predetermined period or at a predetermined times, the update unit 130 increases the basal heart rate.

The update condition here is e.g., the condition at S102 in FIG. 7. Further, for the predetermined period, a period of 90 days or the like may be set. Furthermore, the update unit 130 may count the number of repetition from the start of measurement to the end of measurement of the heart rate information as the predetermined number of times. Specifically, as described above, when the measurement start button is pressed down and then the measurement end button is pressed down, one time may be counted. Alternatively, the predetermined number of times may be the number of times of the determination of the update condition of the basal heart rate. At S102 in FIG. 7, the number of times when comparison processing between the minimum heart rate and the basal heart rate is performed may be employed, and, in this case, the predetermined number of times may be considered as the number of times when the minimum heart rate is obtained. Thus, it may be possible to exclude the cases where the measurement itself is performed, but the minimum heart rate is not obtained from the number of times, and, when the minimum heart rate is obtained at a plurality of times from pressing down of the start button to pressing down of the stop button, count the obtained number of times.

FIG. 12 shows an example of obtaining the minimum heart rate if a determination that the user enters the sleep state during measurement is made. In this case, as shown by the horizontal axis, the predetermined number of times may be considered as the number of sleeping times. In FIG. 12, the values of the minimum heart rate became smaller in the fourth, fifth sleeping times, and update processing of decreasing the basal heart rate was performed. However, during subsequent 90 times, the minimum heart rate was not below the basal heart rate. In this case, it is considered that the current basal heart rate may not reflect the actual condition of the user, i.e., may take excessively small value. Thus, it is preferable to increase the value of the basal heart rate as shown in FIG. 12, and the value is increased by one in FIG. 12. Note that, as described above, the basal heart rate as the reference of the determination processing should not be largely changed, and it is not preferable to excessively increase the basal heart rate when increasing.

In this manner, it may be possible to perform not only the update processing of decreasing but also increasing the basal heart rate. Even in the case where the basal heart rate is lower not to correspond to the user's condition due to environmental changes or the like, it may be possible to perform appropriate update processing. As described above, it is easy to decrease the basal heart rate using the minimum heart rate, and thus, if the update processing of increasing the basal heart rate is inappropriate, it is easy to return the basal heart rate to the appropriate condition.

Further, it may be possible to increase the basal heart rate in consideration of the changes of the basal heart rate or the like due to the age of the user. For example, the update unit 130 may increase the basal heart rate after a lapse of a predetermined period.

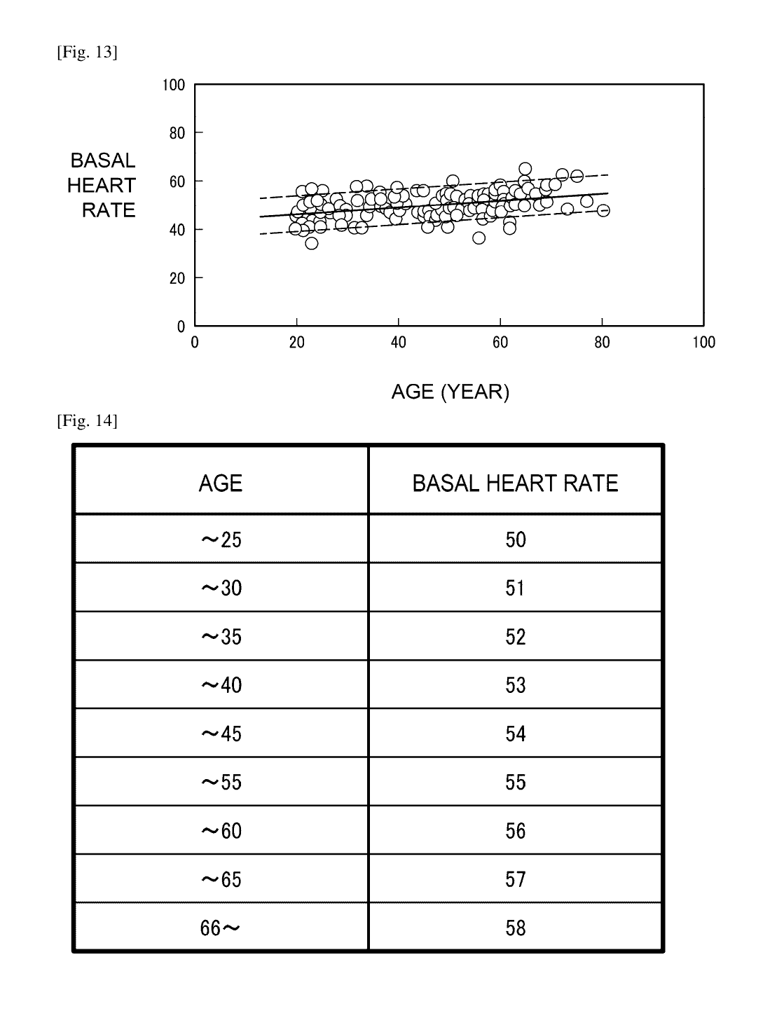

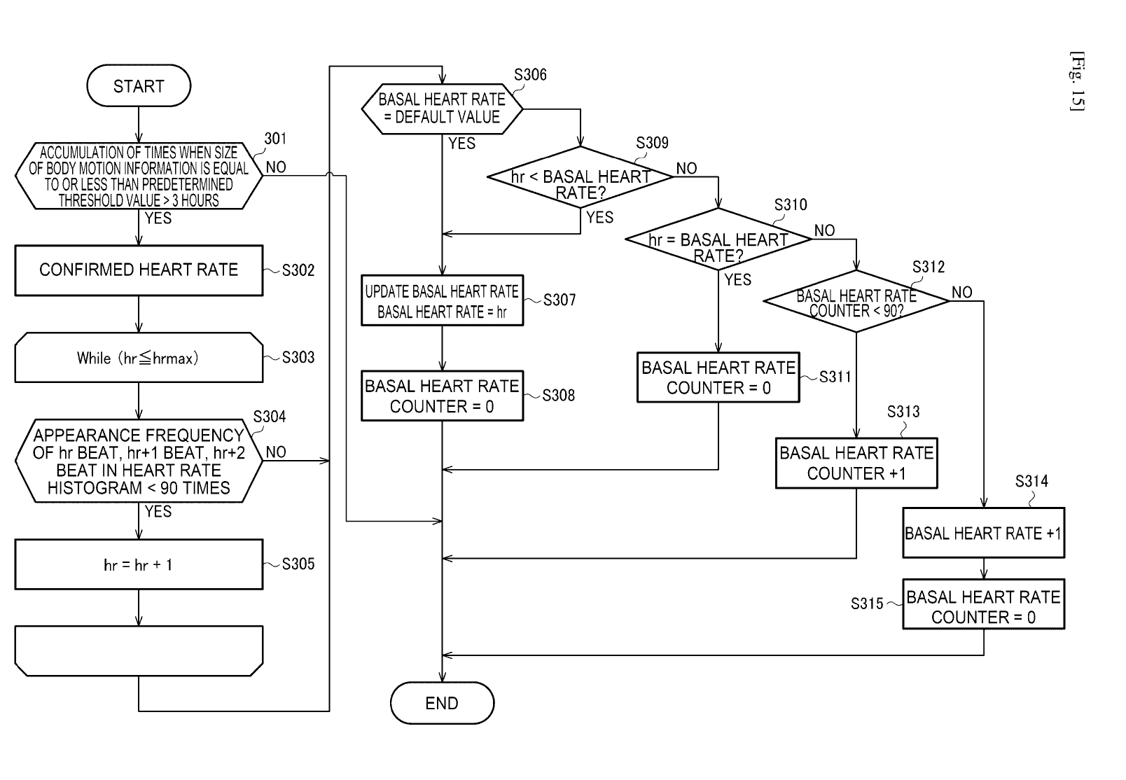

As shown in FIG. 13, the value of the basal heart rate tends to rise with advancing age. That is, if there is no decrease of the basal heart rate due to environmental changes or no decrease of the basal heart rate due to measurement or processing errors, it is considered that the basal heart rate becomes less matched with the user's condition with time. Accordingly, the basal heart rate may be increased in units of e.g., three months, six months, one year. In the example in which the basal heart rate is increased if the update condition is not satisfied, when the update processing is performed during the predetermined period or at the predetermined times, the measurement of the periods or the count of the times are reset, however, in this case, it is not necessary to particularly consider the previous update conditions or the like. Accordingly, regardless of the actually measured heart rate information, the value of the basal heart rate may be reliably increased.

Obviously, it is conceivable that appropriate update processing including variations with age is performed by increasing processing if the update condition is not satisfied, and, in this case, the possibility of excessive increase of the value of the basal heart rate by the update processing with a lapse of the predetermined period is undeniable. However, as described above, it is easy to decrease the value of the basal heart rate that has excessively increased, and this is not problematic. As described above, it is not preferable that the basal heart rate varies at higher frequencies, however, the predetermined period here is assumed as a longer period to some extent, and this is not significantly problematic in consideration of the point.

1.3.3 Setting and Update Processing of Default Value

Next, processing with respect to a default value of the basal heart rate will be explained. As described above, the numerical values of the heart rate information significantly differ among individual users, and, it is necessary to perform comparison processing with a value as reference not an absolute value, i.e., the basal heart rate for appropriate processing. Accordingly, even in a situation in which use of the biological information processing system 100 (e.g., the wearable apparatus 200 in FIG. 4A, etc.) is just started and the heart rate information of the user is not actually measured for obtaining the basal heart rate, some default value may be set as the basal heart rate.

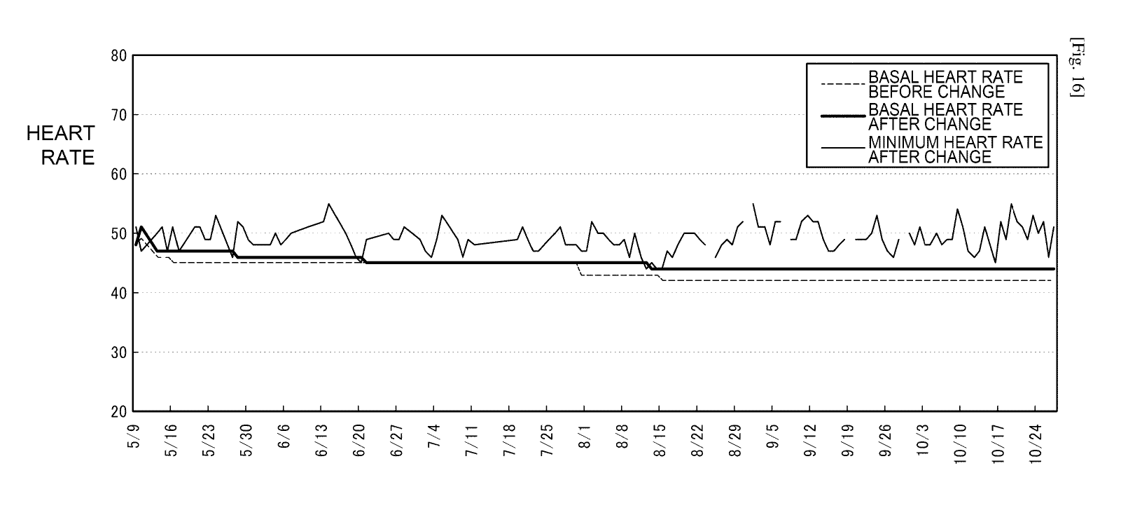

Specifically, regardless of personal data of the user, a predetermined value may be used as the default value. Or, as shown in FIG. 14, a table associating predetermined personal data (e.g., age) and default values of the basal heart rate may be held and the default value may be set from personal data input by the user when the use of the apparatus is started and the table. Or, a formula for obtaining the default value from personal data may be held and the default value may be set from a calculation result using the formula.

However, these default values are set without consideration of differences among individual users. Accordingly, in the embodiment, the value of the basal heart rate is determined based on the heart rate information in the determination unit 120, and thereby, the basal heart rate is changed (updated) using the actual measurement values from the subject user. That is, the processing in the determination unit 120 is update processing of the basal heart rate in a broad sense, however, in the embodiment, the processing in the determination unit 120 and the processing in the update unit 130 may be made different.

Specifically, the determination unit 120 performs processing of replacing the default value of the basal heart rate set by the biological information processing system 100 by the minimum heart rate obtained from the heart rate information, and the update unit 130 performs update processing of updating the basal heart rate determined by the determination unit 120.

As described above, it is not impossible that the update processing in the update unit 130 changes the value of the basal heart rate significantly to some degree because the replacement is performed by the minimum heart rate for decreasing the value. However, as shown in FIG. 12, it is necessary to determine the processing of increasing the value in the predetermined period or at the predetermined number of times, and, as described above, the smaller increase is assumed.

The default value of the basal heart rate is set without consideration of the individual differences, and the value may be largely different from the value that should be actually used. In this case, if the default value is larger than the ideal value, it may be possible to make the value closer to the ideal value in a shorter time even by the same processing as that of the update unit 130. However, if the default value is smaller than the ideal value, the technique of spending a longer time to gradually increase the value is not preferable because a long time may be taken to reach the ideal value.

Accordingly, in the embodiment, when the value is changed from the default value to the value using the actual measurement values, regardless of the magnitude relationship between the minimum heart rate and the basal heart rate (default value), replacement by the minimum heart rate may be performed. In this manner, even in the case where the default value is smaller than the ideal value, it may be possible to make the value closer to the ideal value in a shorter time.

1.3.4 Processing Using Body Motion Signal

Further, in the embodiment, in updating of the basal heart rate, etc., body motion information acquired by the body motion information acquisition unit 140 may be used. Specifically, when the determination that, based on body motion information in a given minimum heart rate measurement period, the minimum heart rate measurement period contains the period in which the smaller body motion of the user is determined is made, the determination unit 120 may employ the minimum heart rate obtained based on the heart rate information in the minimum heart rate measurement period as the basal heart rate.

As described above, the basal heart rate is the heart rate in the deep sleep state or in a state determined to be the deep sleep state, and the lowest or equivalent to the lowest heat rate for the subject user. That is, when the value of the basal heart rate is changed from the default value using the actually measured heart rate information, it is necessary to use the minimum heart rate in the deep sleep state or the like. Particularly, in the processing in the determination unit 120 as described above, even when the minimum heart rate is larger than the basal heart rate (default value), the basal heart rate is replaced by the minimum heart rate. Accordingly, in the processing of the update unit 130 or the like, there is a risk that a very high heart rate that does not at all contribute to the calculation of the basal heart rate (e.g., the heart rate during exercise) is employed as the basal heart rate. Obviously, if the minimum heart rate is subsequently obtained from the low heart rate information during sleep, the basal heart rate is updated by the value and there is no problem. However, the use frequency during sleep may be very low depending on the user, and there is a risk that the excessively high basal heart rate is used in a longer period.

Accordingly, when the minimum heart rate is obtained, the body motion information is obtained with the heart rate information, and whether or not the user enters the sleep state within an object period (minimum heart rate measurement period) based on the body motion information is determined. Then, the basal heart rate is changed from the default value using the minimum heart rate when the period contains the period in the sleep state. On the other hand, if the user is not in the sleep state within the period, the basal heart rate is not changed from the information in the period, and the value of the basal heart rate may be prevented from being excessively larger.

Specifically, as shown at S201 in FIG. 8, the body signals are acquired every several seconds. In this regard, like the heart rate information, moving average processing may be performed to suppress variations. Further, there are some cases where the value of the body motion information is not zero even in an ideal rest state such that the value of gravitational acceleration is detected in the acceleration signal, and, at S201, the difference value (differential value) of the body motion information may be acquired.

Then, at S204, an average value or a summation of the values obtained at S201 in a given period may be calculated and comparison processing with a threshold value may be performed. Various given periods are conceivable here, and e.g., a period of about 10 minutes is used. Through the comparison processing, if the average value is larger than the threshold value, a determination that the body motion of the user is larger and the user is not in the sleep state in the period may be made, and, if the average value is equal to or smaller than the threshold value, a determination that the body motion of the user is smaller and the user is in the sleep state in the period may be made.

Further, in one minimum heart rate measurement period, the times in which the sleep state is determined by the determination are accumulated. Here, if the time in which the sleep state is determined is longer than three hours, in the minimum heart rate measurement period, a determination that the user is in the sleep state and the value that can be used for the processing with respect to the basal heart rate has been obtained is made. On the other hand, if the time in the sleep state is equal to or shorter than three hours, a determination that the user does not enter the sleep state or the user enters the sleep state, but the sleep is not sufficient to be into the deep sleep state is made, and the value is not used for the processing with respect to the basal heart rate.

Furthermore, the body motion information may be used in the update unit 130, and the update unit 130 may determine the update condition of the basal heart rate based on the body motion information. The point that the basal heart rate should ideally be the heart rate in the deep sleep state is the same in the processing in the determination unit 120 and the update processing in the update unit 130, and thus, it may be possible to appropriately update the basal heart rate using information in the period in which the user is in the sleep state at least in a certain amount of time.

More specifically, the update unit 130 may count the number of times at which the body motion of the user is smaller based on the body motion information in the period from the start of measurement to the end of the measurement of the times of repetition as the predetermined number of times.

Thereby, it may be possible to appropriately count the predetermined number of times. For example, when counting one from pressing down of the start button to pressing down of the stop button, the number of times of counting may be very large in a short time by repeated start and stop at a higher frequency in a day depending on the use example by the user. In the extreme example, if ON/OFF are repeated at 90 times in a day, there is a possibility that the basal heart rate is increased in the day.

However, the predetermined number of times should be the number of times when significant update determination is performed. The minimum heart rate corresponding to the condition with sufficiently lower heart rate for the subject user is obtained, the minimum heart rate and the basal heart rate are compared as shown at S102 in FIG. 7, and thereby, a determination that the basal heart rate is updatable is made. If the minimum heart rate is obtained at 90 times during exercise, the possibility that the minimum heart rate is below the basal heart rate is very low, and the processing is not useful in view of updating of the basal heart rate. If the minimum heart rate during exercise or the like is above the basal heart rate continuously at a predetermined number of times, this is a natural result, and increasing the basal heart rate based on the result is not appropriate.

That is, significant determination processing of the update condition is performed in a specified number of times using the body motion information in the counting at the predetermined number of times. Further, even when the significant determination is repeated at a certain number of times, the increasing processing is performed if the minimum heart rate is below the basal heart rate, and thereby, validity to some extent may be secured with respect to the increase of the basal heart rate.

1.3.5 Outline

As the outline of the section, a flowchart showing update processing of the basal heart rate including the above described processing will be explained, and then, differences between the technique of the embodiment and the technique of related art will be explained using graphs based on actual measurement values.

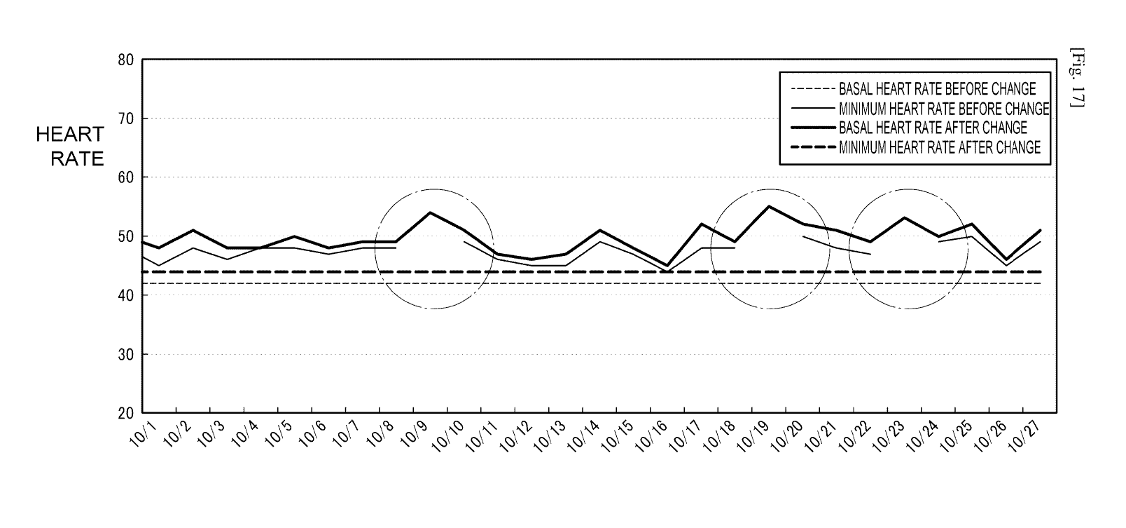

FIG. 15 is a flowchart showing detailed update processing of the basal heart rate. When the processing is started, first, a value formed by accumulation of times in which the average values or integrated values of body motion information (e.g., differential values of acceleration values) in a given period exceed a threshold value of the body motion over the minimum heart rate measurement period is acquired, and whether or not the value exceeds a threshold value of time (e.g., three hours) is determined (S301). The accumulated value of the times is acquired according to the heart rate information or the like as shown at S204 in FIG. 8, and the value may be used.

If No at S301, in the minimum heart rate measurement period as the processing object, the user does not have sufficient sleep and, even if the minimum heart rate is obtained, the minimum heart rate may be determined not to correspond to the basal heart rate. Therefore, in the flowchart of FIG. 15, the calculation processing itself of the minimum heart rate is not performed and the processing is ended.

On the other hand, if YES at S301, it is possible that the user is in the sleep state (particularly, in the deep sleep state), and the calculation processing of the minimum heart rate is performed. In the flowchart of FIG. 15, as described above using FIG. 10, the technique of searching for values with the frequencies continuously exceeding the threshold value from the lower heart rate is assumed, and, first, the start of the search is set as hr=hrmin (S302). The numerical value range smaller than hrmin is not the object of the search, and a value smaller than the lower limit of the numerical value range that can be taken by the basal heart rate may be used for hrmin here. For example, if it is considered that there are few users having the value of the basal heart rate below 30, hrmin=30 may be set.

Then, in a range from hrmin to the upper limit of hrmax of the search range (S303), comparison processing between the frequency of the histogram and the frequency threshold value (e.g., 90) is performed and the minimum heart rate is searched for. A value larger than the upper limit of the numerical value range that can be taken by the basal heart rate may be used for hrmax. Here, the horizontal axis of the histogram is integer values, and whether or not all of the frequency for the heart rate value of hr, the frequency for the heart rate value of hr+1, frequency for the heart rate value of hr+2 are smaller than a threshold value is determined (S304).

If Yes at S304, as in the case of the heart rate=44 or the like in FIG. 10, hr here is determined not to be the minimum heart rate. Accordingly, update processing of increasing hr is performed and the search is continued. Here, hr is incremented (S305) and the loop at S303 is continued.

On the other hand, if No at S304, as in the case of the heart rate=48 in FIG. 10, hr here may be determined to be the minimum heart rate in the minimum heart rate measurement period as the processing object. Accordingly, the processing exits the loop at S303 and moves to determination of the update condition of the basal heart rate using the obtained minimum heart rate.

Specifically, first, whether or not the current basal heart rate is the default value is determined (S306). If Yes at S306, the processing in the determination unit 120 is performed, and the basal heart rate is replaced by the value of the minimum heart rate regardless of the magnitude relationship between the basal heart rate and the minimum heart rate (S307). Then, to start the counting of the predetermined number of times, the value of the counter is reset to zero (S308) and the processing is ended.

On the other hand, if No at S306, i.e., if the basal heart rate is not the default value, but has been already updated using the actual measurement values of the user, the processing in the update unit 130 is performed, and whether or not the minimum heart rate is smaller than the basal heart rate is determined (S309). If Yes at S309, the basal heart rate may be replaced by the minimum heart rate, and the processing at S307 and S308 is performed and the processing is ended.

If No at S309, whether or not the basal heart rate and the minimum heart rate are equal is determined (S310). If the rates are equal, the value of the counter for counting the predetermined number of times is reset (S311). The case of Yes at S310 is the case where, though the update condition of the basal heart rate (the condition at S309) is not satisfied, but the minimum heart rate based on the actual measurement values is equal to the basal heart rate, and thereby, the current basal heart rate may be determined to be appropriate as a value representing the user's condition, in other words, not an excessively small value. In this case, the processing of increasing the basal heart rate is unnecessary in the immediate future, and the counting of the predetermined number of times may be restarted from zero. Note that, in implementation, a modification in which S310 and S311 are omitted and the determination at S309 is based on hr<=basal heart rate may be made.

If No at S310, the minimum heart rate is larger than the basal heart rate, and the update processing of decreasing the value is unnecessary. Accordingly, whether or not to perform the processing of increasing the basal heart rate is determined. Specifically, whether or not the counter for counting the predetermined number of times is less than a given threshold value (here, 90) is determined (S312). If Yes at S312, the number of times when the update condition is not satisfied does not reach the predetermined number of times, and the basal heart rate is not changed and the value of the counter is incremented (S313). If No at S312, the update condition is not satisfied at the predetermined number of times, and the basal heart rate is increased. Here, the value of the basal heart rate is increased by one (S314). After the processing at S314, for restarting the count of the predetermined number of times, the value of the counter is reset to zero (S315) and the processing is ended.

Next, FIGS. 16 and 17 show the differences in the basal heart rate, etc. between the technique of related art and the technique of the embodiment. FIG. 16 shows a graph when the minimum heart rate is obtained by the technique of related art and the basal heart rate is updated and a graph when the minimum heart rate is obtained by the technique of the embodiment and the basal heart rate is updated with respect to log data of the same heart rate information. As clearly known from the drawing, in the technique of related art, the calculation accuracy of the minimum heart rate is insufficient and the basal heart rate excessively decreases on May 16, August 1, August 15, however, according to the technique of the embodiment, it may be possible to keep the basal heart rate at an appropriate level.

Further, FIG. 17 shows graphs of the basal heart rate and the minimum heart rate according to the technique of related art and the basal heart rate and the minimum heart rate according to the technique of the embodiment. In the range of FIG. 17, there are no variations of the basal heart rate, however, like FIG. 16, update processing of excessively decreasing the basal heart rate is suppressed in the technique of the embodiment, and the basal heart rate of the embodiment has larger values than those of the technique of related art. Further, from comparison between the minimum heart rate in the technique of related art and the minimum heart rate of the embodiment, in most measurements, the minimum heart rates of the embodiment have larger values than those in the technique of related art, and it is known that the possibility that the minimum heart rate is calculated to be excessively lower may be suppressed.

Further, when the minimum heart rate is larger than the basal heart rate, there is a risk that a false determination that the user is not in the sleep state is made. The sleep state determination here is based on the heart rate information and different from the technique using body motion information like S301 in FIG. 15. FIG. 17 shows the example in which the minimum heart rate itself is not obtained if the sleep state is not determined. The basal heart rate is lower in the technique of related art, and, as shown in broken-line circles, when there is a day on which the minimum heart rate is higher, a determination that the user is not in the sleep state is made even if the user is sleeping. On the other hand, in the technique of the embodiment, the basal heart rate may be kept at the appropriate level, and, as shown in the broken-line circles, even on a day on which the minimum heart rate is higher, the possibility of the false determination of the state of the user may be suppressed.

### 1.4 Generation Processing of Health Degree Information Using Basal Heart Rate

A specific example of processing of health degree information using the basal heart rate will be explained. As below, calculation techniques of respective information will be explained, and then, examples of display screens used when the obtained health degree information is presented to the user will be explained. Note that the health degree information obtained based on the basal heart rate is not limited to the following.

1.4.1 Calculation Technique of Calorie Consumption

As described above, in the technique of related art, VO₂ is estimated based on the formula (1) from the heart rate (HR), the maximum VO₂ (VO₂ₘ), the maximum HR (HRₘ), the resting VO₂ (VO₂ᵣ), and the resting HR (HRᵣ), and the minute energy (calorie) consumption EE (EE=VO₂*5/1000 kcal) is obtained. However, VO₂ₘ, VO₂ᵣ, HRₘ, HRᵣ are the values without consideration of individual differences, and VO₂ₘ and HRₘ are not actually measurable, the influence by activity (ACT) is not taken in consideration, and their reliability is lower. Particularly, in consideration of application of monitoring of the health condition of the user in a longer period (e.g., full day) at body motion or at non-body motion or the like, the problem of the technique using the formula (1) is significant.

Accordingly, in the embodiment, the calorie consumption is calculated with reference to the above described basal heart rate HR₀. Specifically, the formula is converted into the following formula (2) using the minute energy consumption EE₀ corresponding to the basal heart rate HR₀. EE in the formula (2) is the minute calorie consumption, EE₀ is EE in the basal state, and EEₘ is the maximum value of EE.

$$\begin{matrix}[ Math . 2 ] & \\ \frac{( EE - {EE}_{0} )}{( {EE}_{m} - {EE}_{0} )} = \frac{( HR - {HR}_{0} )}{( {HR}_{m} - {HR}_{0} )} & ( 2 )\end{matrix}$$

Further, the formula (2) is solved for EE, and the following formula (3) is obtained.

$$\begin{matrix}[ Math . 3 ] & \\ EE = {EE}_{0} + \frac{( {EE}_{m} / {EE}_{0} - 1 )}{( {HR}_{m} / {HR}_{0} - 1 )} \times \frac{{EE}_{0}}{{HR}_{0}} \times ( HR - {HR}_{0} ) \\ suppose that \\ \frac{( {EE}_{m} / {EE}_{0} - 1 )}{( {HR}_{m} / {HR}_{0} - 1 )} = x , HR - {HR}_{0} = Δ HR \\ EE = {EE}_{0} + x \times \frac{{EE}_{0}}{{HR}_{0}} \times Δ HR \\ further , suppose that \\ EE - {EE}_{0} = Δ EE \\ Δ EE = x \times \frac{{EE}_{0}}{{HR}_{0}} \times Δ HR & ( 3 )\end{matrix}$$

Here, EE₀ is a value corresponding to the minute basal metabolic rate of the user and it is known that the basal metabolic rate BM per day can be calculated by various techniques, and EE₀ may be obtained in advance. Further, HR₀ can be determined from the actual measurement values as described above, and a value of heart rate information actually measured at the time may be used for HR. Therefore, in the formula (3), if the value of the part shown by x is determined, the calorie consumption (minute calorie consumption EE) may be obtained.

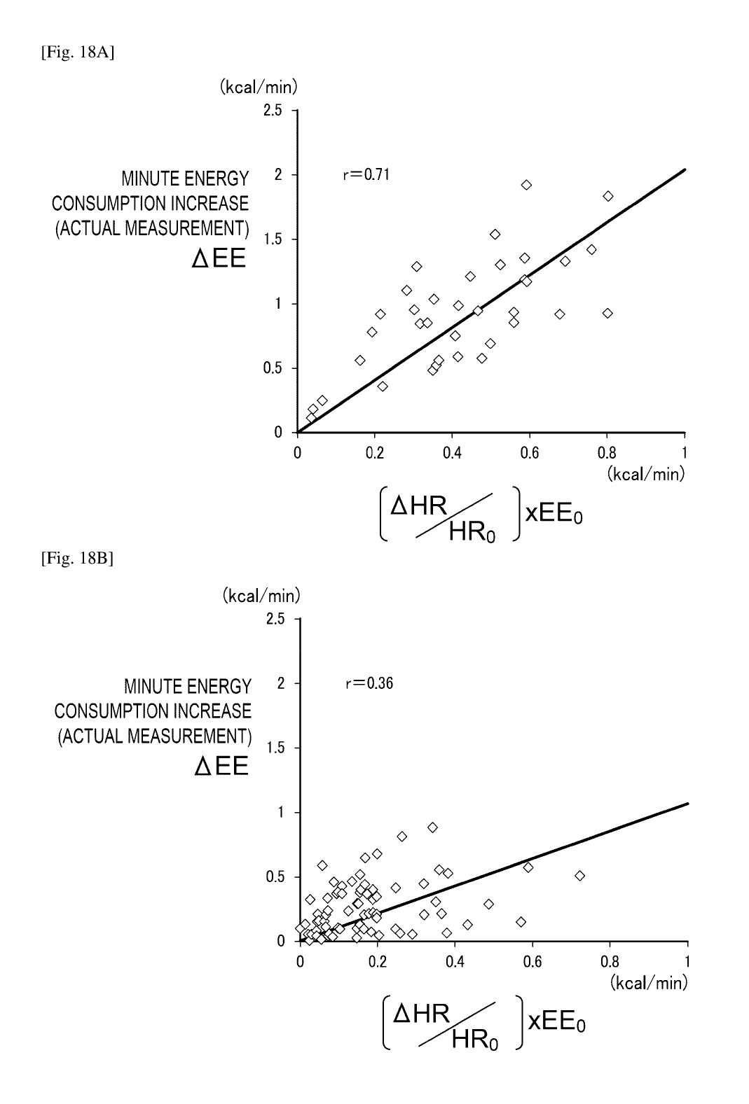

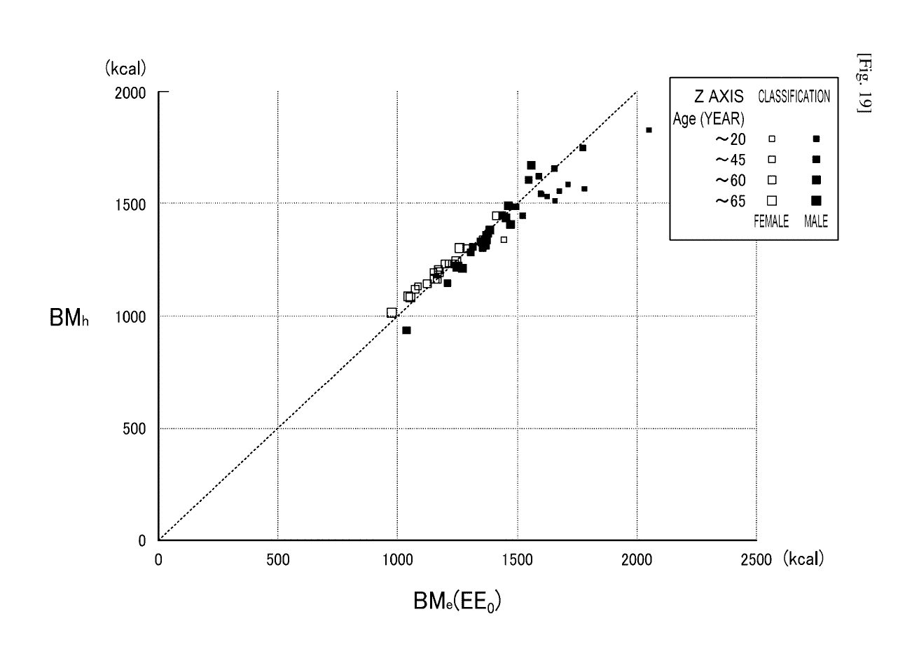

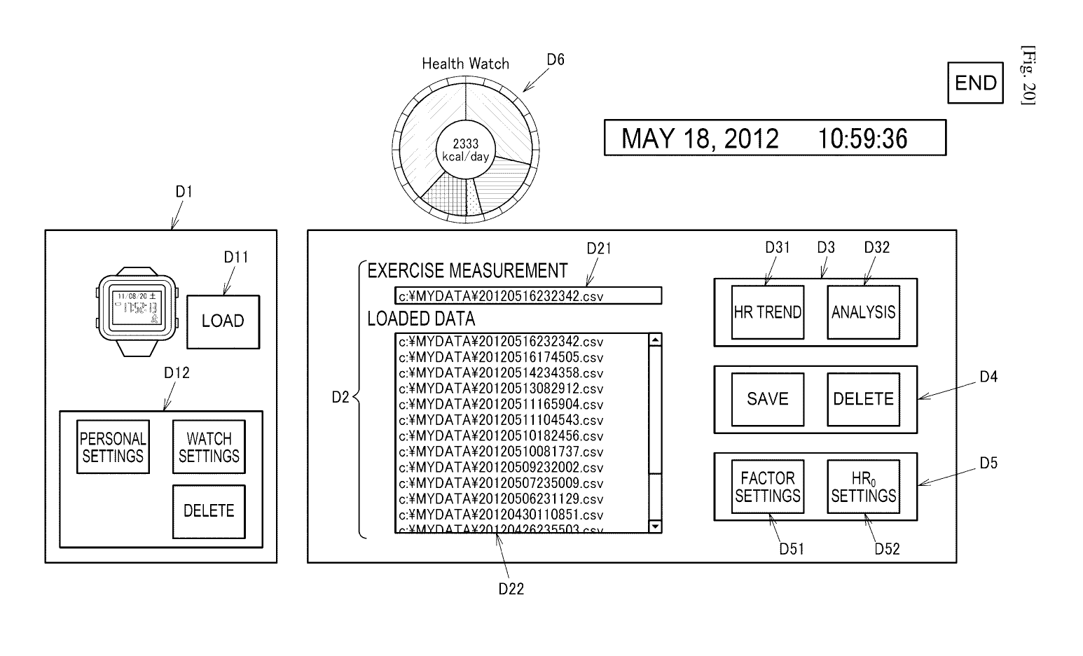

Here, FIGS. 18A and 18B show graphs formed by plotting values with DELTA_EE in the formula (3) along the vertical axis and (DELTA_HR/HR₀) EE₀ in the formula (3) along the horizontal axis. FIG. 18A corresponds to the plot of the values during activity (also described as during exercise or at body motion) and FIG. 18B corresponds to the plot of the values during non-activity (also described as at rest or at non-body motion, and specifically, in resting recumbent position, seated position, standing position, or the like).

As is known from the values along the vertical axis and the horizontal axis and the formula (3), when the plotted points are linearly approximated, the gradient of the line represents the factor x. In comparison between FIGS. 18A and 18B, the gradient of the line of FIG. 18A corresponding to the factor at body motion is larger than the gradient of the line of FIG. 18B corresponding to the factor at non-body motion. This shows that the value of the factor x is larger at activity than that at non-activity, and, when the energy consumption (calorie consumption) is obtained, it is necessary to perform calculation with the factor changed in response to activity or non-activity.

Further, in the formula (3), as an index value DELTA_HR indicating the degree of variations with respect to the reference value of the heart rate information, HR−HR₀ is used. That is, the reference value is the basal heart rate HR₀. However, during wakefulness, regardless of body motion or non-body motion, the user often takes the standing position or the seated position. In this case, baroreceptor sensitivity acts and, even if both physical activities and mental activities are very weak, the heart rate increases compared to HR₀. Therefore, actually, as the reference value of the heart rate information when DELTA_HR is determined, not HR₀ itself, but a value in consideration of the increased amount is desirably used. Accordingly, in the embodiment, factors ALPHA and BETA equal to or more than one are set, and DELTA_HR=HR−ALPHA_HR₀ is used at body motion and DELTA_HR=HR−BETA_HR₀ is used at non-body motion.

As described above, in consideration of the point that the factor x should be set to different values at body motion and at non-body motion and the point that, as DELTA_HR, not HR−HR₀, but the increased amounts ALPHA and BETA of the reference value should be used, the applicant proposes the following formula (4) and the following formula (5) as the calculation formulae of calorie consumption. The formula (4) is a formula for calculation of the calorie consumption at body motion and the formula (5) is a formula for calculation of the calorie consumption at non-body motion.

$$\begin{matrix}[ Math . 4 ] & \\ EE = {EE}_{0} + x \times ( HR - α {HR}_{0} ) \times \frac{{EE}_{0}}{{HR}_{0}} & ( 4 ) \\ [ Math . 5 ] & \\ EE = {EE}_{0} + y \times ( HR - β {HR}_{0} ) \times \frac{{EE}_{0}}{{HR}_{0}} & ( 5 )\end{matrix}$$

As described above using FIGS. 18A and 18B, the factor x is used at body motion and the factor y is used at non-body motion. This may be due to the difference that x indicates a correction factor of SV (SV represents stroke volume as a blood volume output per beat) increase at body motion, i.e., mainly by muscular exercise and y indicates a correction factor of SV increase at non-body motion, i.e., by mental activities or body position variations. That is, the minute calorie consumption (EE) is proportional to the cardiac output (CO) (EE proportional to CO=SV*HR), however, the different factors x, y are respectively used because the increase of SV with respect to the increase of HR is different between at body motion and at non-body motion.

Further, as described above, ALPHA is for correction of the resting heart rate in wakefulness at body motion. Here, it has been experimentally confirmed that, even at rest, the heart rate increases about 1.2 times the value HR₀ of the basal heart rate due to wakefulness and the standing position. Therefore, for ALPHA in the embodiment, ALPHA=1.2 is used as a normal value. On the other hand. BETA is for correction of the initial value before mental activities or the body position variations at non-body motion, however, this state is considered as being closer to the rest state and BETA=1.0 is used as a normal value in the embodiment.

Furthermore, as described above, in order to obtain the calorie consumption from the formulae (4), (5), it is necessary to determine the value of the minute calorie consumption EE₀ corresponding to the basal heart rate HR₀ in advance. A value per minute is obtained from the basal metabolic rate BM per day and the obtained value corresponds to EE₀. For a technique for obtaining BM, a widely known Harris-Benedict Equation may be used.

Note that, in the embodiment, EE₀ may be obtained based on the minute cardiac output CO (CO is a product of the heart rate HR and the stroke volume SV). Oxygen binds to hemoglobin in lungs, is transported by the heart, released and used in tissues (the brain and muscles), and the rest is eliminated in the lungs, and VO₂ and the minute cardiac output CO have a proportional relationship (VO₂ proportional to CO=HR*SV proportional to EE).

The applicant has found a principle that, at the slow-move sleep phase of brain wave (during deep sleep), the heart rate takes the basal heart rate HR₀ and the cardiac index CI at the time (suppose that the body surface area is BSA, CI=CO/BSA) is a value with little difference among individuals. Further, the applicant has created the following formula (6) for obtaining the cardiac output during sleep (CO₀) as value with little difference among individuals by application of the principle.

CO₀=6.9*Age^{-0.25}*BSA (6)

Oxygen consumption may be estimated from the formula (6) and standard hemoglobin content in blood (male 15 g/dl, female 13.5 g/dl), the difference between oxygen saturation in arterial blood (97.5%) and oxygen saturation in venous blood (75%), and the volume of oxygen binding to hemoglobin of 1 g (1.34 ml) and EE may be estimated from VO₂, and similarly, the basal metabolic rate BM may be estimated from the oxygen consumption corresponding to CO₀.

Specifically, suppose that the hemoglobin content in blood is Hb, by the calculation using the respective values and including the values for unit conversion, the basal metabolic rate estimate BM is obtained by the following formula (7). By substitution of a specific value to Hb, the male basal metabolic rate estimate Bₘ, and the female basal metabolic rate estimate BM_{f} are expressed by the following formulae (8).

BM=CO₀*Hb*1.34*(0.975−0.75)*10*60*24*5/1000 (7)

Male BMₘ=325.6*CO₀

Female BM_{f}=293.0*CO₀ (8)

FIG. 19 shows a graph with the basal metabolic rate obtained by the Harris-Benedict Equation in related art along the vertical axis and the basal metabolic rate estimate obtained by the formulae (6) and (8) along the horizontal axis with respect to subjects of various ages and sexes. In this case, the correlation factor is r=0.96 and they have a very high correlation. That is, the basal metabolic rate (and the minute calorie consumption EE₀) may be accurately calculated by the technique using the formulae (6) and (8) proposed by the applicant.

Next, techniques of determining x in the formula (4) and y in the formula (5) will be explained. Specifically, given standard values may be used or the values may be obtained from actual measurement values when exercise with a fixed load is performed.

First, the technique using the standard values is explained. As expressed in the formula (3), x takes a value closer to (EEₘ/EE₀−1)/(HRₘ/HR₀−1). It is known that, statistically, EEₘ takes values of 49−0.29Age for male and 41−0.33Age for female from age (Age) from 20 to 70 and HRm takes a value of 220−Age. Further, EE₀ may be obtained from the above described BMₘ or BM_{f}, and HR may be acquired using the actual measurement values from the heart rate sensor or the like.

By calculation of x using these values, an average was a value closer to a value of 4.8 plus/minus 1.5 (standard deviation SD) (about 5). Therefore, when x is unknown, the value of 5 is used as the standard value. Note that the value obtained by measurement of HR and VO₂ at mental activities and statistic calculation is used for the value of y, and 1.5 is used as the standard value of y in the embodiment.

Note that, in the above described technique of determining x, an approximate value of x is obtained using the statistical values for EEₘ and HRₘ that are difficult to be actually measured. Accordingly, like the technique of PTL 1 shown in the formula (1), etc., a problem that dealing with the differences among individual users is difficult is left.

In the embodiment, the value of x may be obtained from the actual measurement values. Specifically, the following formula (9) is obtained by transforming the formula (4).

$$\begin{matrix}[ Math . 6 ] & \\ x = ( EE - {EE}_{0} ) \times \frac{1}{( HR - α {HR}_{0} ) \times \frac{{EE}_{0}}{{HR}_{0}}} & ( 9 )\end{matrix}$$

HR₀ and EE₀ on the right side may be obtained by the above described techniques, and it is experimentally known that 1.2 is preferably used for ALPHA. Further, the heart rate information HR is obtained from the heart rate sensor or the like, and thus, if the value of EE may be obtained, it may be possible to determine x from the actual measurement values. Here, in view of the purpose to obtain the minute calorie consumption EE of the technique of the embodiment, it may be impossible to determine the value of EE in advance if arbitrary activity states are objects (including the body motion time and the non-body motion time). However, if the object is limited to the exercise with known predetermined exercise load, it may be possible to calculate the calorie consumption EE by the exercise in advance. For example, it is known that, when stepping exercise of two steps per second is continued for three minutes (exercise of about 3 Mets), the minute calorie consumption EE satisfies the following formula (10).

EE=3*1.05*weight/60 (10)

As described above, in the case where an instruction to perform predetermined exercise may be given to the user, all of the values of HR₀, EE₀, ALPHA, HR, EE may be determined and acquired, and x may be obtained from the actual measurement values from the formula (9).

1.4.2 Calculation of Deep Sleep Time Information

It is known that lack of sleep (e.g., the case where the deep sleep time is four hours or less) has a significant effect on the autonomic nerves of the following day and has an adverse effect on the health, and the sleep time is an important index value in evaluations of lifestyles. Particularly, the time in the deep sleep state (or described as brain wave slow-wave sleep state) in the deeper sleep of the sleeping states or the like is important as the index value indicating the sleep state. For example, the case where the sleep time itself is longer, but the deep sleep time is shorter has an adverse effect on the health and leads to subjective symptoms such that you have not recovered from fatigue though you were supposed to sleep.

Accordingly, in the embodiment, information as to whether or not the user is in the deep sleep state (in the strict sense, information on the deep sleep time as the time in the deep sleep state of 24 hours) is calculated as the health degree information.

As described above, in the deep sleep state, the value of the heart rate information HR takes a value closer to the value of the basal heart rate HR₀. Accordingly, in comparison between HR and HR₀, whether or not the user is in the deep sleep state may be determined. Note that, even in the deep sleep state, the value of HR varies, and it is conceivable that the value of HR is larger than HR₀. Therefore, the value used for the comparison with HR is not the HR₀ itself, but a margin to some extent is provided and a value of HR₀*(sleep factor) is used. That is, if the following formula (11) holds, the user is determined to be in the deep sleep state, and the integrated value of the times when the formula (11) holds of 24 hours is used as the deep sleep time. Here, the sleep factor of the formula (11) differs for each user, and, for example, a statistically obtained value of 1.12 or the like may be used.

HR<=HR₀*(sleep factor) (11)

1.4.3 Calculation of Stress Information

Stress information representing load on the user may be used as an index value indicating the health degree. Here, as the stress information, physical stress due to physical activities at body motion and mental stress due to mental activities at non-body motion are conceivable.

The degree of load by the physical stress and the mental stress is significantly reflected in the value of the heart rate information HR. Here, it is known that the heart rate increases at non-body motion mainly because of brain activity, and thus, the mental stress may be evaluated by integrating the times when the heart rate rises at a certain level or more at non-body motion. A stress factor is provided as the indication, and, if HR satisfies the following formula (12), a determination that mental stress to the extent to be watched is applied to the user is made and the integrated value of the times when the formula (12) holds is used as an index value (mental stress information) with respect to the mental stress.

HR>=HR₀*(stress factor) (12)

Here, the value of the stress factor differs among individuals, and may be externally input. Note that, in the case where the stress factor is unknown, reduction of operation load by the user is intended, or the like, the stress factor=1.8 or the like may be used as a statistically obtained value.

On the other hand, the physical stress represents the load on the user at body motion, and the value may be obtained in consideration of the heart rate increase mainly by the muscle activities. Specifically, like the above described mental stress, determination using the formula (12) may be performed, and, the physical stress is different in the point that the body motion time is the object.

Note that, various techniques of determining the body motion time or the non-body motion time are conceivable, and, for example, processing may be performed based on the sensor information from the body motion sensor. When the body motion sensor is an acceleration sensor, if the acceleration detection value as the sensor information from the sensor is larger, the body motion time may be determined and, if the acceleration detection value is smaller than that at body motion, the non-body motion time may be determined. Alternatively, not the magnitude of the acceleration detection value itself, but the frequency characteristics (e.g., corresponding to pitches at walking, running exercises) are obtained and the body motion time or the non-body motion time may be determined from them. That is, it is only necessary that the body motion sensor of the embodiment is a sensor that may determine the body motion time or the non-body motion time in the calculation of the stress information, and the acceleration sensor or another sensor may be used. Further, any technique of determining the body motion time or the non-body motion time based on the sensor information may be employed.

Thus obtained stress information may be used as an index value for determination that, the smaller value of the integrated time is more preferable with respect to the mental stress and the more moderate value (larger at the level not causing lack of exercise and smaller at the level not causing excessive load) of the integrated time is more preferable with respect to the physical stress.

1.4.4 Display Control

As described above, in the techniques of the embodiment, the calorie consumption, the deep sleep time, and the stress information may be acquired as the health degree information. It is conceivable that, with the acquired health degree information, the health condition of the user may not be easily understood only by simple display of the values. Accordingly, in the embodiment, the acquired health degree information is presented in an easily understandable form at a glance for the user (or an attending doctor of the user, a health adviser, or the like) using a technique of graphic representation or the like.

As below, specific examples of display screens will be explained using FIGS. 20 to 24, and the formats of the display screens in the embodiment are not limited to those. Further, in view of presentation of a certain volume of information at once, the screens in FIG. 20, etc. are assumed to be displayed on the portable terminal apparatus 300 in FIG. 6 or the like. However, in the case where the wearable apparatus 200 has a display unit in a sufficient size, the display screen is simplified, or the like, the display screens for information presentation may be displayed on the display unit of the wearable apparatus 200.

FIG. 20 shows an example of a home screen displayed when the wearable apparatus 200 and the portable terminal apparatus 300 or the like is connected. In the home screen, cover information including personal information input mode for age, sex, height, weight, ID, data file management, input/output management via communication, basal heart rate (HR₀) settings, and initial factor settings is displayed. As below, their specific explanation will be made.

In an area of D1 in FIG. 20, settings, etc. with respect to the wearable apparatus 200 are made. Specifically, under a condition that the wearable apparatus 200 and the portable terminal apparatus 300 are connected, when a load button of D11 is pressed, information acquired in the wearable apparatus 200 is loaded into the portable terminal apparatus 300. The loaded information may be only information as results of the calculation based on the HR and HR₀ such as the calorie consumption, the deep sleep times, and the stress information, or all of sensor information of the heart rate sensor 10 and the body motion sensor 20, and various modifications may be made.

In D12, registration of user information, watch setting, etc. may be performed. The detailed explanation of the function of D12 will be omitted.

D2 shows the file names for saving the information loaded by the load button of D11. Specifically, the latest data file may be displayed in D21 and the data files loaded in the past may be displayed in D22.

D3 is buttons for displaying temporal changes of the heart rate information (HR trends), analysis results based on the information, etc. of the acquired information (here, on the assumption that all of the HR values have been acquired). The display screen examples when the buttons are pressed will be described later.

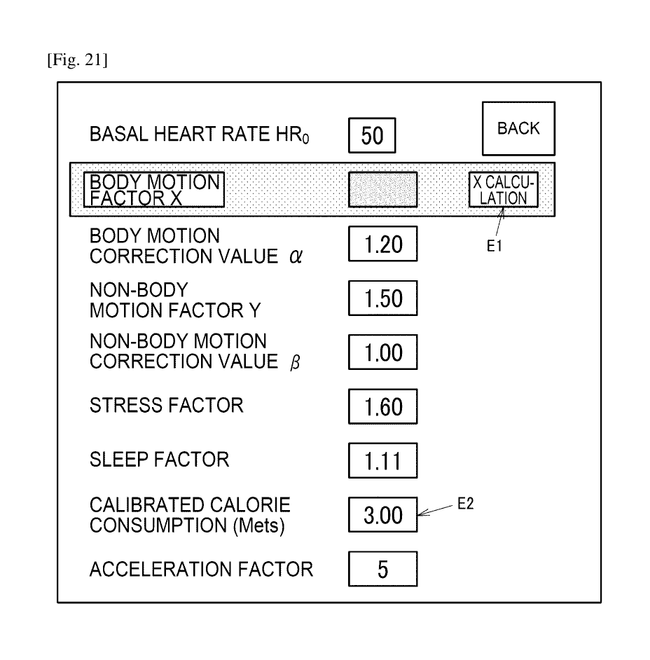

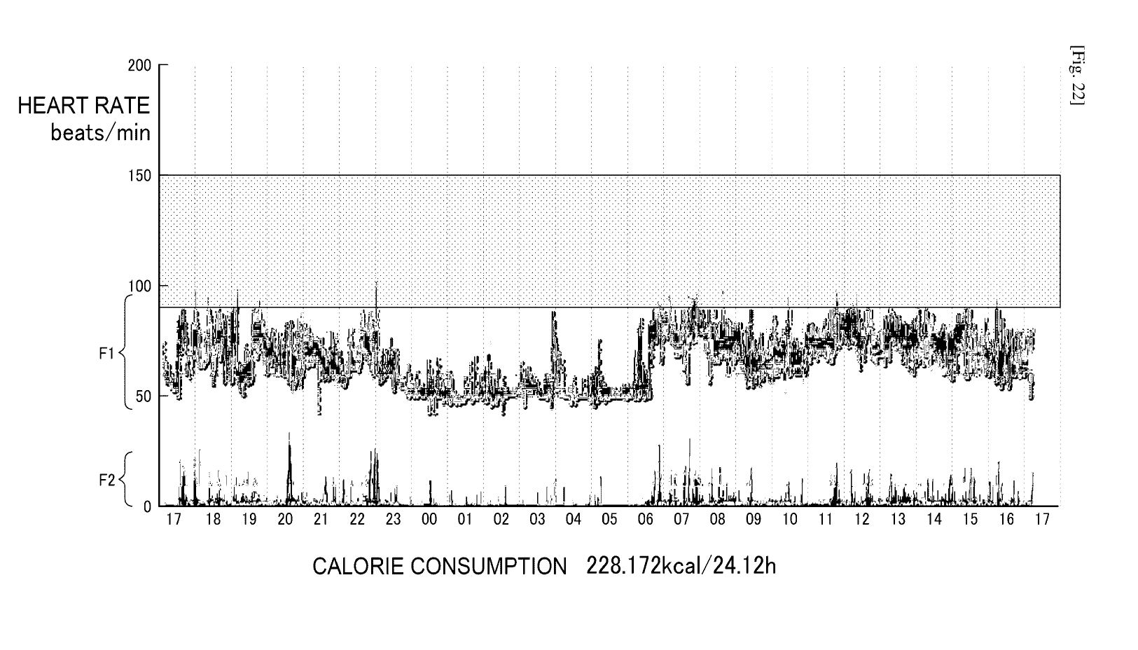

D4 is buttons for instruction to save and delete the data files. D5 is an area for advance preparation in the calculation of the health degree information. Specifically, D51 is a button for calling up a screen for setting factors for obtaining the health degree information, and, when D51 is pressed, the screen is changed to the screen in FIG. 21. In FIG. 21, the value of HR0, the factors used for the calculation of the calorie consumption such as x, y, ALPHA, BETA, or the sleep factor used for the calculation of the deep sleep time, the stress factor used for the calculation of the stress information, etc. are set. Further, x may be set from the actual measurement values as described above, and, in this case, the user may press the x calculation button shown by E1 and start given exercise. The value of the load of the exercise performed at setting of x from the actual measurement values (in the above described example, stepping exercise of two steps per second is continued for three minutes), and this corresponds to calibrated calorie consumption (Mets) of E2. The acceleration factor is a value representing the threshold value of the acceleration detection value when the body motion time or the non-body motion time is determined when the acceleration sensor is used as the body motion sensor. Note that the value of the acceleration factor differs depending on the range of the acceleration sensor or the like, and the unit is not a with reference to the standard gravitational acceleration or m/s².

D52 is a button used when HR₀ is set. As described above, HR₀ is updated using the heart rate information, however, when the button of D52 is pressed down, setting processing of HR₀ is performed because the case where resetting is performed according to an explicit instruction by the user or the like is conceivable.

FIG. 22 shows an example of a screen displayed when the HR trend button of D31 in FIG. 20 is pressed. FIG. 22 shows temporal changes of the value of the heart rate information HR in continuous 24 hours and temporal changes of the calorie consumption calculated based on HR and HR₀. In FIG. 22, the graph shown by F1 shows the temporal changes of HR and the graph shown by F2 shows the temporal changes of calorie consumption. From FIG. 22, biological information (lifestyle habit information) of the user such that the user is in the sleep state from about midnight to 6:30 is known.

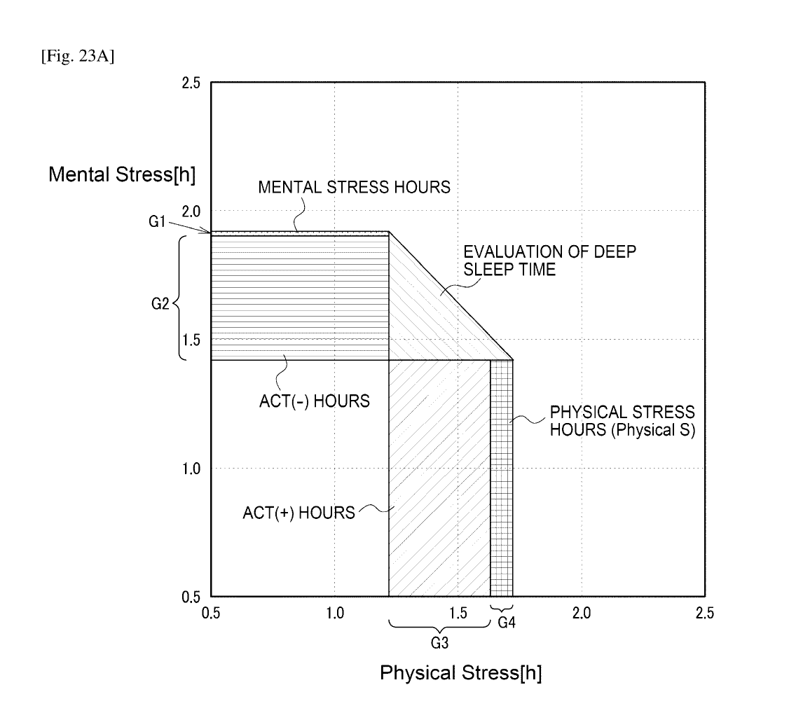

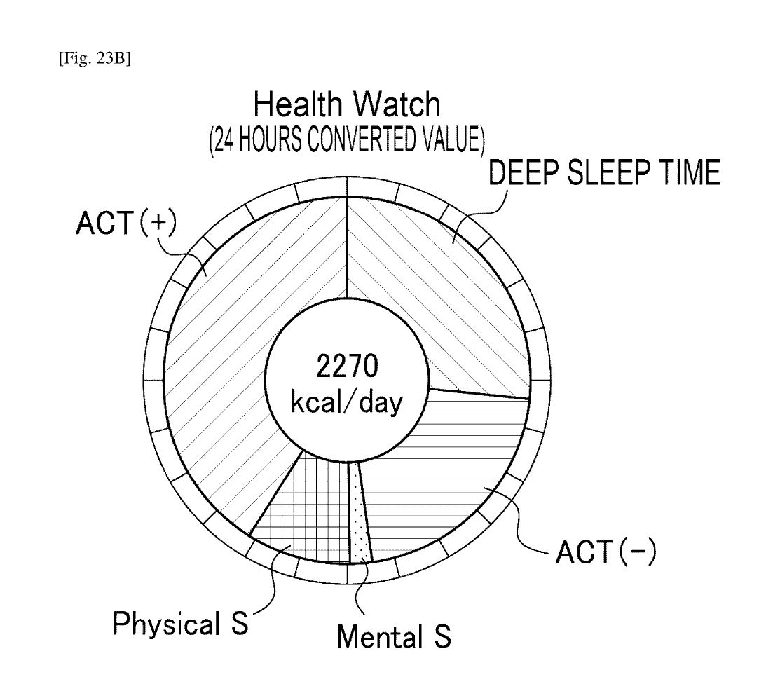

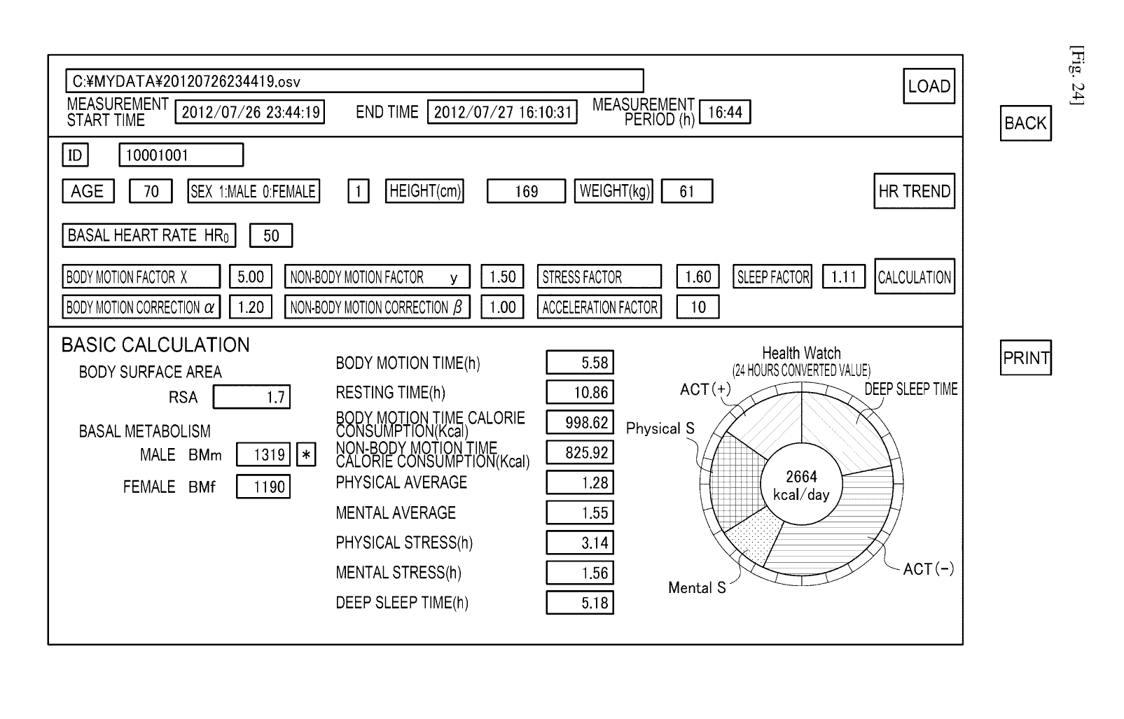

It is desirable to present the health degree information collectively in an easily understandable manner. In the embodiment, display screens in FIGS. 23A and 23B may be displayed in the area shown by D6 in FIG. 20 (FIG. 20 shows the example in which the screen in FIG. 23B is displayed).

FIG. 23B is a graph collectively representing the calorie consumption, the deep sleep time, the stress information in a day. The deep sleep time in FIG. 23B is the time when the user is in the deep sleep state, ACT (−) is the non-body motion time without mental stress. Mental S shows the non-body motion time with mental stress. Further, Physical S shows the body motion time with physical stress, and ACT (+) shows the body motion time without physical stress. Further, the calorie consumption for 24 hours is displayed in the center part of the circular graph.

Using FIG. 23B, the ratio of the deep sleep time, the time with mental stress, the time with physical stress, etc. of 24 hours may be intuitively understood. Specifically, the healthy life style is considered as a condition in which a sufficient rest (sleep) is taken, physical activities (physical stress) is sufficient, the mental stress is less, and the calorie intake and the calorie consumption are balanced. FIG. 23B is seen in such a viewpoint, and thereby, the health condition of the user may be easily grasped.

Note that the case where the wearable apparatus 200 or the like of the embodiment is not worn for continuous 24 hours is conceivable in consideration of charging of the apparatus or the like. In this case, FIG. 23B is the presentation of the relative relations among the respective times in the easy-to-see form, and presents data not reaching 24 hours converted to 24 hours (e.g., when the wearing time is 12 hours, processing of doubling the values of the respective times or the like is performed). Accordingly, the actual times may be difficult to be understood.

Therefore, without conversion to the basis of 24 hours, a technique of displaying the actual times as they are may be employed as shown in FIG. 23A. In FIG. 23A, both the vertical axis and the horizontal axis indicate times (in the unit of hour). G1 shows the actual time of the mental stress. G2 shows the actual time of the non-motion time (without feeling of mental stress), G3 shows the actual time of the motion time (without feeling of mental stress), and G4 shows the actual time of the physical stress.

Further, the deep sleep time may be represented by the triangular area in FIG. 23A. In this case, it is assumed that the deep sleep time is represented not by the area of the triangular area, but color or the like. For example, different colors may be used such as green for sufficient deep sleep time (seven hours or more), yellow for less deep sleep time (from four to seven hours), and red for clearly lack of deep sleep time (four hours or less).

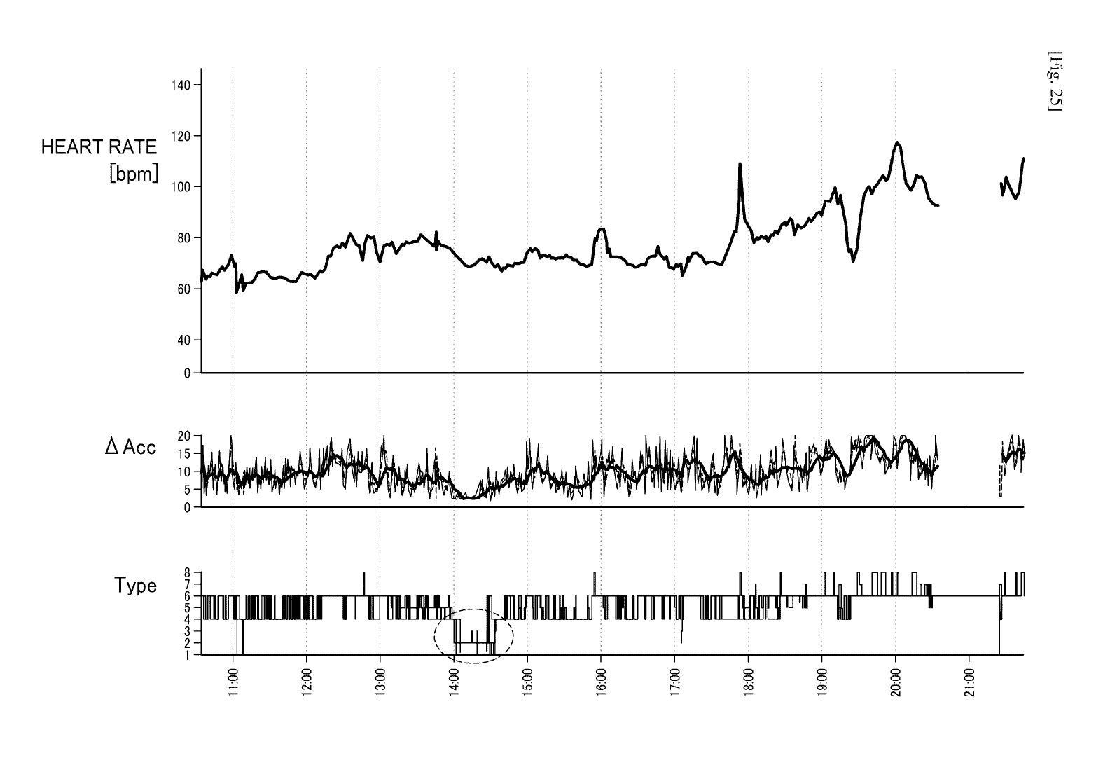

Furthermore, the graph representations in FIGS. 23A, 23B, etc. are difficult for grasping accurate values though intuitively and easily understandable. Accordingly, when the analysis button shown in A32 of FIG. 20 is pressed, an analysis screen shown in FIG. 24 may be displayed. In the analysis screen, for example, as shown in FIG. 24, the personal information of the user and the parameters used for the calculation of the health degree information are displayed, and specific values of the actually measured health degree information are displayed. In this regard, the graph in FIG. 23B or the like may be displayed at the same time. The analysis screens in FIG. 24, etc. are displayed, and thereby, more accurate values may be known.

### 2. Second Embodiment

### 2.1 Outline of Second Embodiment

First, a technique of the embodiment will be explained. As described above, it is preferable to set the reference value when the processing using the heart rate is performed. PTL 3 discloses an example using the basal resting pulse rate (basal pulse rate) and PTL 4 discloses an example using the basal resting pulse rate, the base pulse rate, etc.

Here, the basal resting pulse rate and the resting pulse rate are the pulse rates when the user is at rest and corresponds to the resting heart rate in the embodiment. Further, the base pulse rate of PTL 4 is the pulse rate after the user awakens and before rising. Furthermore, as will be described later, the basal heart rate in the embodiment refers to the lowest heart rate during sleep in definition.

That is, if the heart rate as the reference of the processing is directly measured, the measurement should be performed when the user is in the corresponding state. For example, “user is at rest” corresponds to the case where the seated position state is continued for a fixed time or more, etc., and, in order to measure the resting heart rate, it is necessary to attach an electronic apparatus for measurement in an operating condition in the states. Further, like the basal heart rate of the embodiment and the base pulse rate of PTL 4, if the heart rate in the sleep state or immediately after the change from the sleep state to the awake state or the like, for example, it is necessary to attach the electronic apparatus in the operating condition during sleep.

The applicant assumes that an electronic apparatus with reduced size, weight, and power consumption is realized, and thereby, continuous measurement for a longer period is performed, i.e., the electronic apparatus is used for acquisition of lifelog. If the electronic apparatus is attached in the operating condition from the awake state to the sleep state as assumed, the above described information may be measured.

However, the utilization form of the biological information processing system (or the electronic apparatus including the system) differs among users. Accordingly, depending on the lifestyle or the like of the user, it may be difficult to directly measure desired information.

In the case where the wrist-watch (band-shaped) electronic apparatus as described above using FIGS. 4A and 4B is used, some users attach the electronic apparatuses only in predetermined periods for the various reasons that the user dislikes wrist-watches or the user has a delicate skin and develops a rash due to squeezing by the band and attachment of sweat. For example, in the use case where the electronic apparatus is attached away from home and detached at home, it is impossible to acquire information in the sleep state. Accordingly, it is difficult to directly measure the basal heart rate in the embodiment.

On the other hand, some users do not attach the electronic apparatuses during the day because the apparatuses hinder in relations with work or the like and attach the electronic apparatuses only during sleep. In this case, it is difficult to measure the resting heart rate corresponding to the information in the awake state with very little motion (rest state).

Particularly, the use cases often depend on the lifestyles of users, and a situation that, after purchase of the electronic apparatus, the use is continued without single acquisition of information in the sleep state may occur. There is no problem if the measured heart rate value is output as it is, however, in the case where various kinds of processing using the heart rate such as sleep determination and calorie consumption determination, which will be described later, is performed if it is impossible to acquire the basal heart rate or the resting heart rate as reference, it is impossible to perform the processing in the first place.

Furthermore, the basal heart rate and the resting heart rate differ among individuals, and, if the initial value obtained from the age, etc., is used, there is a risk that an unreasonable determination result is output. For example, FIG. 25 shows data of the case where an initial value (=57) obtained from the age, etc. is set as the basal heart rate because the measurement in the sleep state is not performed. In the case of FIG. 25, the original basal heart rate of the user is about 45 and the initial value larger than the actual value is set, however, as described above, it is impossible to update the initial value using the actual measurement values because attachment in the sleep state is not performed. As a result, the determination that should be made with reference to 45 is performed with reference to 57, and the determination result is inappropriate.

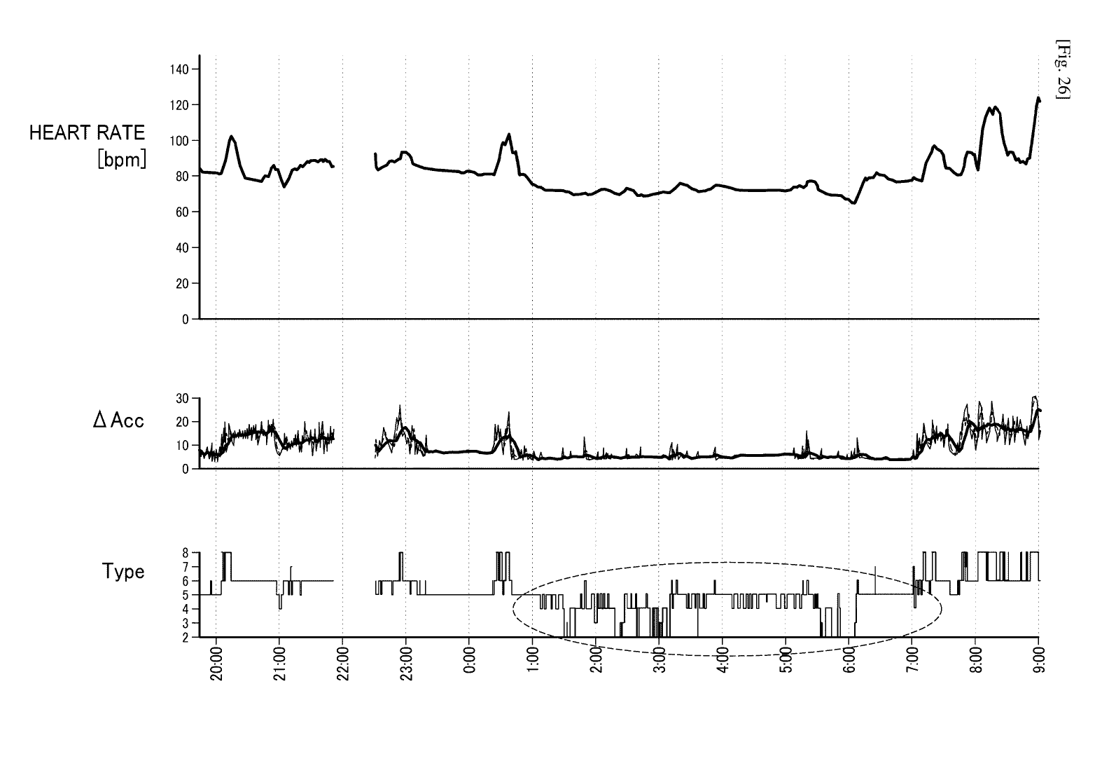

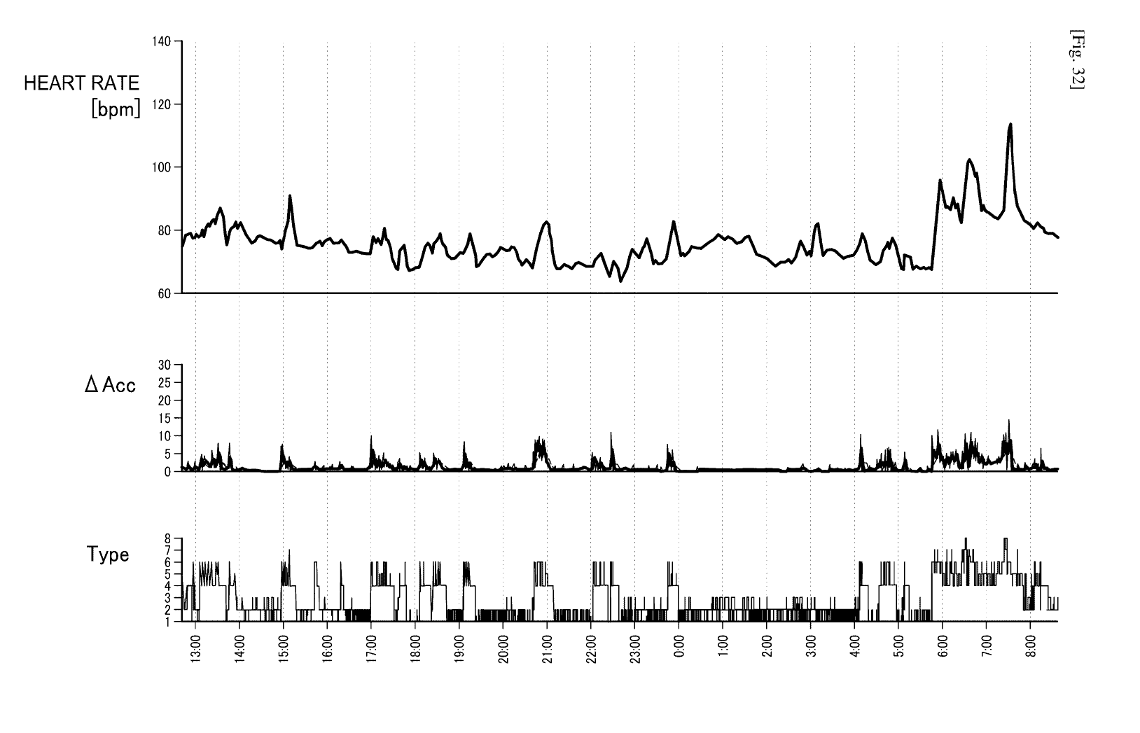

FIG. 25 shows the measurement data during the midday hours, and it is known that the user is constantly in the awake state within the period. However, in the case where the determination of the sleep state using the heart rate is performed as expressed in the above described formula (11) using a threshold value Th satisfying Th=basal heart rate*ALPHA (ALPHA is constant larger than one), Th is excessively larger with the initial value. Accordingly, as shown by a broken circle in FIG. 25, there are some cases where the measured heart rate is below Th, and a false determination that the user is in the sleep state is made though the user is actually in the awake state in the part. Note that the middle part of FIG. 25 shows amounts of changes of body motion information (acceleration signal) and the lower part of FIG. 25 shows information representing the states of the user obtained from the basal heart rate, etc. Specifically, in the lower part of FIG. 25, TYPE 7-8 show states with high load, TYPE 5-6 show activity states, TYPE 4 shows a relaxation state, TYPE 1-3 show sleep states. FIG. 25 shows the example in more detailed classification such that TYPE 1 shows deep sleep and TYPE 2 shows light sleep, however, here, the detailed explanation is omitted. Further, this applies to FIGS. 26 and 32.

In contrast, FIG. 26 shows the case where the user's original basal heart rate=69 is used and an initial value (=55) smaller than that is set. In this case, the threshold value Th is smaller than the value that should be used. FIG. 26 shows measurement data during night hours, and it is known that the user is in the sleep state at least from 1:00 to 7:00 in the period. However, Th is larger, and the measured heart rate may be above Th as shown by a broken circle in FIG. 26, and a false determination that the user is in the awake state is made though the user is actually in the sleep state in the part. Note that it is possible to measure the information in the sleep state in FIG. 26, and the basal heart rate may be measured and made closer to the value that should be set.

Further, this applies to the resting heart rate, and, if the value is not set in consideration of differences among individuals, reliability of the processing using the resting heart rate is lower. For example, in the case where the calorie consumption is calculated using the resting heart rate as will be described later, the calculated calorie does not coincide with the actual consumption.

As described above, there are some cases where it is difficult to directly measure the basal heart rate and the resting heart rate depending on lifestyles or the like though the heart rates should be set using actually measured information for dealing with differences among individuals. On the other hand, the techniques of related art of PTL 3, PTL 4, etc. do not disclose any techniques for dealing with the cases.

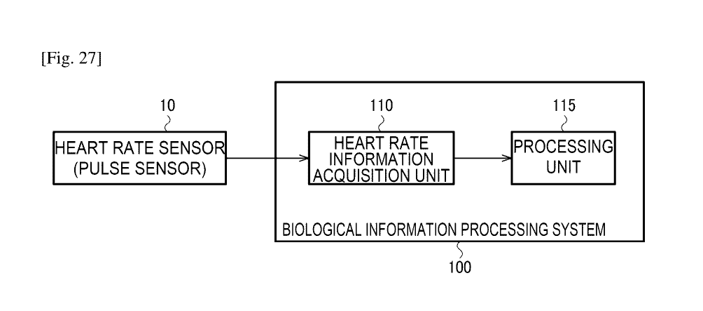

Accordingly, the applicant proposes a technique of calculating the information based on actual measurement values even when it is impossible to directly measure the basal heart rate or the resting heart rate due to the lifestyle or the like of the user. Specifically, as shown in FIG. 27, the biological information processing system 100 (biological information processing apparatus) according to the embodiment includes a heart rate information acquisition unit 110 that acquires heart rate information of a user, and a processing unit 115 that Obtains a resting heart rate as the heart rate information determined to correspond to a rest state of the user based on the heart rate information and calculates a basal heart rate based on the resting heart rate.

Here, the basal heart rate represents the heart rate information when the user is in a deep sleep state. In the deep sleep state, variations due to mental activities are not caused, and it may be possible to accurately perform processing using the heart rate infortnation not only at exercise (body motion) but also at rest (non-body motion).

In this manner, the basal heart rate may be obtained regardless of the lifestyle or the like of the user. For example, for the user not attaching the electronic apparatus in the sleep state, if the resting heart rate is obtained in the awake state, it may be possible to calculate (estimate) the basal heart rate from the resting heart rate. Further, the resting heart rate is obtained from the actual measurement values and the calculated basal heart rate reflects the characteristics of the user as a measuring object, and, compared to the basal heart rate uniformly set from the age, etc., it may be possible to perform processing with high accuracy in consideration of differences among individuals.

Note that, when the basal heart rate is calculated from the resting heart rate, the processing unit 115 may calculate the basal heart rate by performing calculation processing using a given calculation formula with respect to the resting heart rate, and more specifically, may use the following formula (13), which will be described later.

Further, the basal heart rate may be directly measured depending on the user, however, the resting heart rate may not be directly measured. Therefore, the biological information processing system 100 according to the embodiment may include a heart rate information acquisition unit 110 that acquires heart rate information of a user, and a processing unit 115 that obtains a basal heart rate as the heart rate information determined to correspond to a deep sleep state of the user based on the heart rate information and calculates a resting heart rate based on the obtained basal heart rate. In this case, for example, the processing unit 115 may calculate the resting heart rate by performing calculation processing using a given calculation formula with respect to the basal heart rate, and more specifically, may use the following formula (14), which will be described later.

In this manner, for the user attaching the electronic apparatus only in the sleep state, it may be possible to calculate (estimate) the resting heart rate using the basal heart rate obtained from the measurement result in the sleeping state. Further, the resting heart rate reflects the differences among individuals like the above described example.

Note that, in the second embodiment, a technique of not performing sleep determination at least at the stage of obtaining the basal heart rate and resting heart rate will be explained. Specifically, the heart rate information is classified in response to levels of body motion information (acceleration signal) and histograms are created, and the resting heart rate is obtained from the histogram corresponding to the middle acceleration level and a second heart rate is obtained from the histogram corresponding to the low acceleration level. Further, a first heart rate is calculated from the resting heart rate and the final basal heart rate is obtained by comparison between the first heart rate and the second heart rate.

In this case, if the heart rate information in the sleep state has not been acquired, it is impossible to directly measure the basal heart rate. Specifically, it is assumed that it is impossible to sufficiently acquire the heart rate information at the low acceleration level or obtain the second heart rate. However, in this case, the resting heart rate must have been appropriately obtained, and the first heart rate calculated from that is expected to take a reasonable value for the user. That is, appropriate processing may be performed without the determination of the sleeping state at the stage of obtaining the basal heart rate.

On the other hand, in the third embodiment, a technique with sleep determination will be explained. In this case, using the result of the sleep determination, it may be possible to determine whether the value obtained using a histogram or the like is a value corresponding to the basal heart rate or a value corresponding to the resting heart rate. In this case, unlike the second embodiment, processing of comparing plural candidates of basal heart rates may be omitted. Note that, in the third embodiment, an example of calculating not only the basal heart rate from the resting heart rate but also the resting heart rate from the basal heart rate will be explained.

Further, generally, it is supposed that there are some differences in heart rate between the awake state and the sleep state, however, among the users, there are examples having smaller differences. As a modified example of the third embodiment, a technique of appropriately setting the basal heart rate, etc. for the users will be explained.

### 2.2 System Configuration Example

A configuration example of the biological information processing system 100 according to the embodiment is as shown in FIG. 27, and the processing unit 115 may include the determination unit 120 and the update unit 130 shown in FIG. 1, for example. Further, as shown in FIGS. 2 and 3, the biological information processing system may include the body motion sensor 20, the body motion information acquisition unit 140, the health degree information calculation unit 150, the alarm unit 210, the communication unit 220, etc. In addition, various modifications may be made to the configuration of the biological information processing system 100 like the first embodiment, and the detailed explanation will be omitted.

### 2.3 Flow of Processing

Next, a flow of processing of the embodiment is explained. The processing of obtaining the basal heart rate and the resting heart rate is performed basically when single measurement over a given measurement period is ended based on the heart rate information acquired in the measurement period. Here, for example, the measurement period may be a period from detection of pressing down of a start button to detection of pressing down of a stop button by the user. Alternatively, if it is possible to determine the attachment state of the electronic apparatus, the time when the electronic apparatus is attached may be set to the start point of the measurement period and the time when the electronic apparatus is detached may be set to the end point of the measurement period. In addition, various modifications may be made to definition of “single measurement”, “measurement period”. Further, as below, the processing is performed in units of single measurement, however, a modification in which processing is performed in combination of a plurality of measurements or the like may be made.

In the measurement period, the heart rate information acquisition unit 110 acquires heart rate information at predetermined intervals, and, similarly, the body motion information acquisition unit 140 acquires body motion information at predetermined intervals. The predetermined interval is e.g. about four seconds. That is, when the measurement period is T seconds, in the measurement period, about TA values of heart rate information and corresponding values of body motion information are acquired. Further, processing of calculating moving average of the acquired body motion information or the like may be performed at the predetermined intervals.

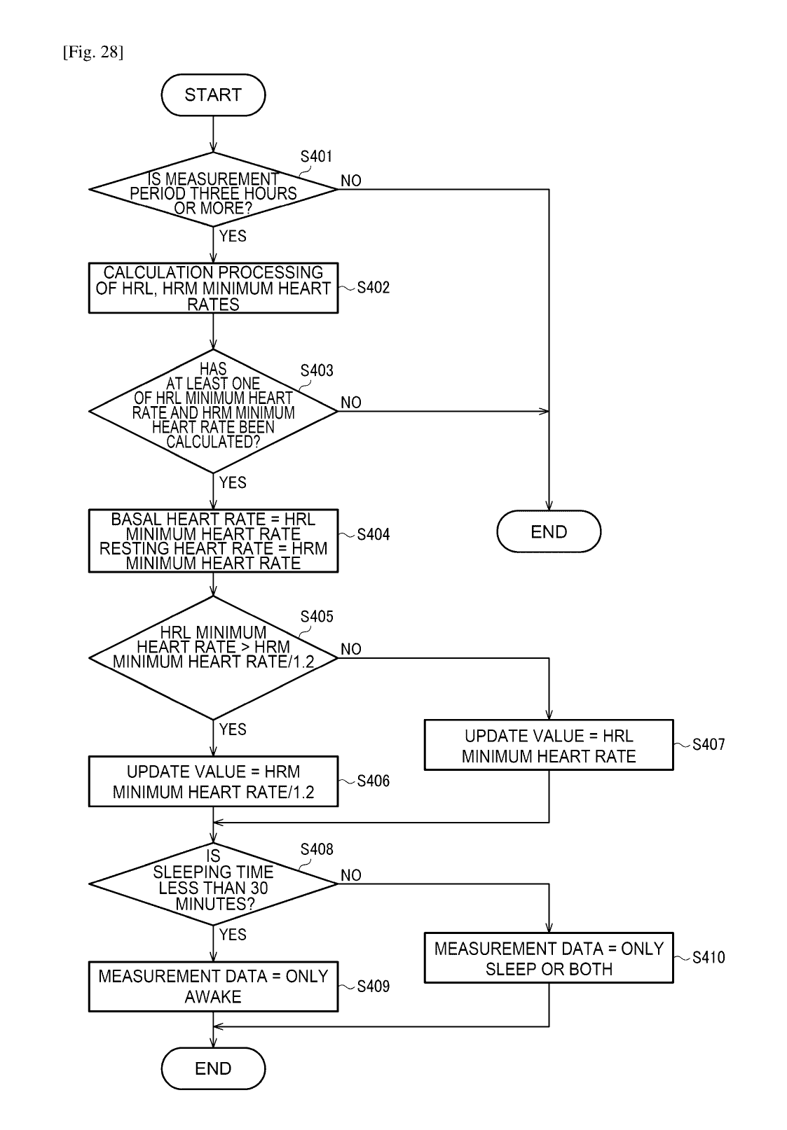

On the above described assumption, the processing of the embodiment is explained using a flowchart in FIG. 28. When the processing is started, first, whether or not the measurement period is three or more hours is determined (S401). If No at S401, a determination that the measurement is not performed in the sufficient period is made, the basal heart rate or the resting heart rate is not obtained, and the processing is ended. In the embodiment, as will be described later, the calculation accuracy of the minimum heart rate is improved using a histogram, etc. However, if the number of pieces of data is insufficient, there is a risk that a singular value generated due to some error is employed as the minimum heart rate or the like and that leads to reduction in accuracy. Accordingly, in the embodiment, the determination at S401 is performed, and the sufficient measurement period is employed as a condition for executing the processing of obtaining the basal heart rate, etc.

If Yes at S401, calculation processing of an HRL minimum heart rate and an HRM minimum heart rate is performed (S402). Here, the HRL minimum heart rate is information obtained from heart rate information at the low signal level (hereinafter, the acceleration level is taken as an example) of the body motion information, and the HRM minimum heart rate is information obtained from heart rate information at the middle signal level. The details of the HRL minimum heart rate and the HRM minimum heart rate will be described later.

Then, whether or not at least one of the HRL minimum heart rate and the HRM minimum heart rate has been obtained is determined (S403), and, if No, i.e., neither has been obtained, the processing is ended. If at least one has been obtained, the HRL minimum heart rate is employed as the basal heart rate and the HRM minimum heart rate is employed as the resting heart rate (S404). Note that, if the HRL minimum heart rate and the HRM minimum heart rate are not obtained, larger values that may not be taken as the basal heart rate and the resting heart rate (e.g., 255 if eight bits are assigned or the like) are set.

Here, in the sleep state, the acceleration level is very low and, in the exercise state or the like, the acceleration level is very high. In contrast, it is considered that, in the awake state and rest state, the acceleration level is lower than that in the exercise state or the like. Here, assuming that the acceleration level in the rest state is higher than that in the sleep level, the acceleration level in the rest state is a middle level. That is, the HRL minimum heart rate may be considered as the minimum heart rate obtained from the heart rate information in the sleep state. On the other hand, the HRM minimum heart rate may be considered as the minimum heart rate obtained from the heart rate information in the rest state.

Note that, in the embodiment, for simplification of the processing, the acceleration level in the rest state is set to the middle level, however, it is considered that the acceleration level in the actual rest state is as low as that in the sleep state. That is, the processing with the acceleration level in the rest state at the middle level is in the embodiment, and processing employing the HRL minimum heart rate as the resting heart rate as the third embodiment to be described later may be performed.

Through the processing at S404, tentative values are set as the basal heart rate and the resting heart rate. However, in the embodiment, unless whether or not the measurement has been performed in the sleep state is determined, whether or not the heart rate information in the measurement period contains information corresponding to the sleep state is unknown. That is, the processing using the basal heart rate obtained at S404 (e.g., processing of updating the basal heart rate that has been held) should not be performed without conditions.

Accordingly, in the embodiment, the basal heart rate is calculated from the resting heart rate (HRM minimum heart rate) using the following formula (13), and comparison processing between the basal heart rate obtained by the calculation (first heart rate) and the basal heart rate directly obtained (HRL minimum heart rate, second heart rate) is performed.

basal heart rate=resting heart rate/1.2 (13)

Specifically, the comparison processing is comparison processing of the magnitude relationship between the first heart rate (HRM minimum heart rate/1.2) and the second heart rate (HRL minimum heart rate) (S405). Then, if the first heart rate is smaller, i.e., if Yes at S405, the first heart rate is employed as the basal heart rate in the measurement period as the processing object (S406). If not, the HRL minimum heart rate as the second heart rate is employed as the basal heart rate in the measurement period as the processing object (S407). Here, if at least one of the HRL minimum heart rate and the HRM minimum heart rate has not been obtained, the minimum heart rate that has not been obtained described above takes the very large value. Accordingly, if only one has been obtained, the value employed at S406 or S407 is the obtained value (or a value calculated from the obtained value).

Note that the value obtained at S406 or S407 is just the basal heart rate in the measurement period as the processing object, and not necessarily an appropriate value as the basal heart rate. For example, the value of the heart rate information is not constantly the basal heart rate even in the sleep state, but often larger values than the basal heart rate. Accordingly, it may be possible that the basal heart rate obtained from the measurement period is larger than the actual basal heart rate, that is, the appropriate basal heart rate has not been obtained from the measurement period.

Therefore, though not illustrated in FIG. 28, the basal heart rate is not replaced by the update value obtained at S406 or S407 as it is, but whether or not the basal heart rate is updated and, if updated, to what value the basal heart rate is updated may be determined using the held basal heart rate and the update value. This applies to the basal heart rate and the resting heart rate in the third embodiment, and the basal heart rate, etc. obtained from the heart rate information in the measurement period is information used for update processing.

Note that, in the embodiment, in the processing of obtaining the basal heart rate, whether or not the heart rate information contains information corresponding to the sleep state is not determined, and the determination of the sleep state in other processing is not hindered. For example, as shown in FIG. 28, after the basal heart rate is obtained, whether or not the sleeping time within the measurement period is less than 30 minutes may be performed (S408). If Yes at S408, a determination that the heart rate information acquired in the measurement period is only information corresponding to the awake state may be made (S409), and, if No, a determination that the heart rate information acquired in the measurement period contains information corresponding to the sleep state may be made (S410).

### 2.4 HRM, HRL

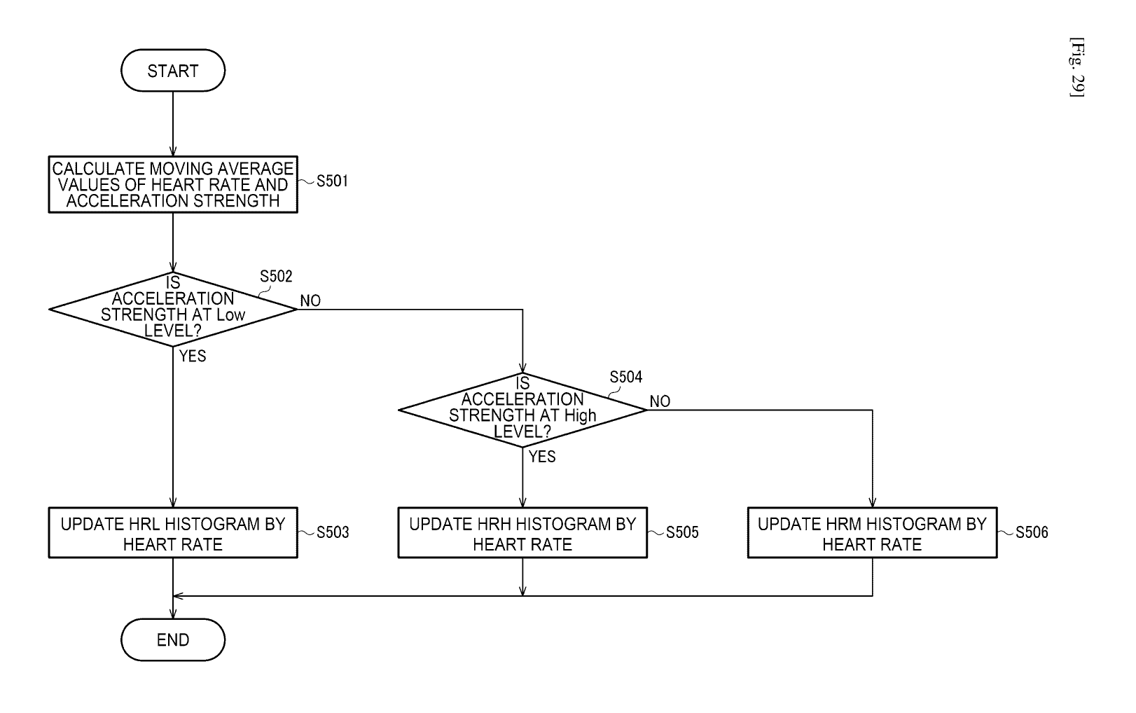

Next, the HRM minimum heart rate and the HRL minimum heart rate will be explained using a flowchart in FIG. 29, etc. As described above, the HRM minimum heart rate has the middle acceleration level and may be considered as the resting heart rate, and the HRL minimum heart rate has the low acceleration level and may be considered as the basal heart rate.

In the embodiment, the heart rate information acquired in the measurement period is classified according to the levels of body motion information. Specifically, the processing shown in the flowchart of FIG. 29 may be performed at each acquisition of heart rate information and corresponding body motion information. The heart rate information, etc. are acquired at predetermined intervals of four seconds or the like, and, also, the processing in FIG. 29 is executed at the predetermined intervals.

When the processing in FIG. 29 is started, first, moving averages of the heart rate information and the acceleration level are calculated (S501). The reason for calculation of the moving averages will be described later. Then, whether or not the acceleration strength (acceleration level) is Low level is determined (S502). In the processing at S502, for example, whether or not the acceleration level is less than a given Low level threshold value AccL may be determined.

If Yes at S502, the acquired heart rate information is information at the low acceleration level, and update processing of an HRL histogram is performed using the heart rate information (S503). The HRL histogram is a histogram of the heart rate information corresponding to the low acceleration level.

If No at S502, whether or not the acceleration strength is High level is determined (S504). S504 may be realized like S502 by determining whether or not the acceleration level is larger than a given High level threshold value AccH. If Yes at S504, the acquired heart rate information is information at the high acceleration level, and update processing of an HRH histogram as a histogram at the high acceleration level is performed (S505).

On the other hand, if No at S504, the acquired heart rate information is information at the middle acceleration level, and update processing of an HRM histogram as a histogram at the middle acceleration level is performed (S506).

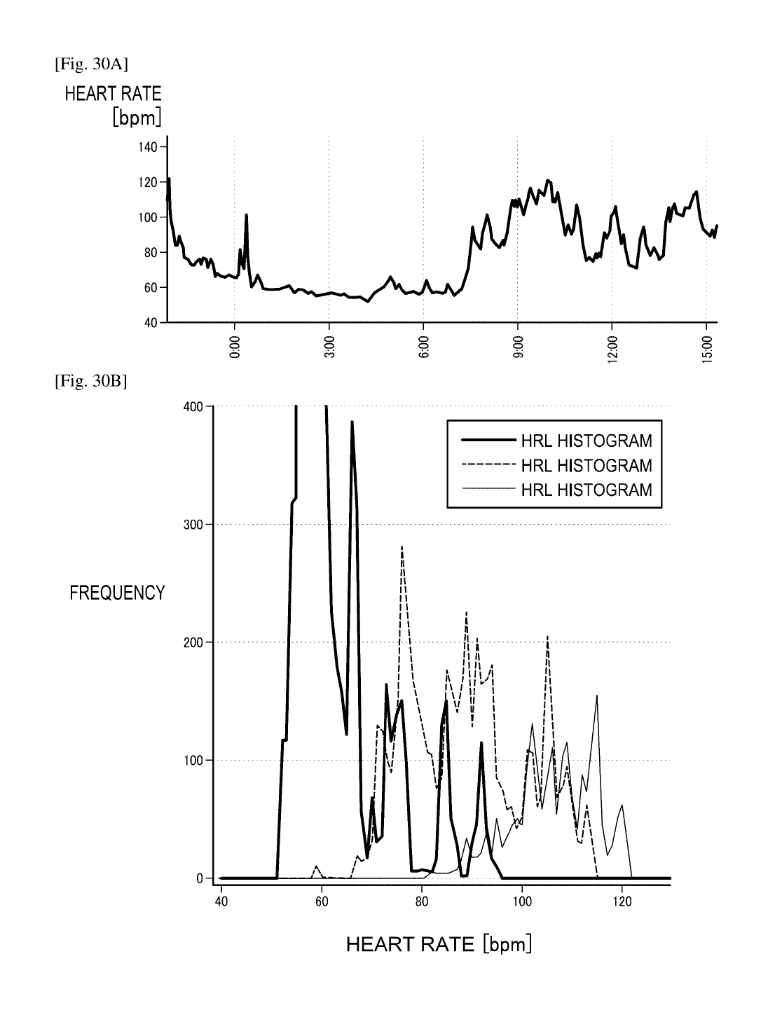

The above described processing is executed over the measurement period, and thereby, the heart rate information may be classified based on body motion information and histograms may be created with respect to each classification. Then, the minimum heart rate corresponding to each classification may be obtained from the created histogram. FIG. 30A shows the actually measured heart rate information and FIG. 30B shows the respective histograms for HRL, HRM, HRH created based on the heart rate information.

However, the heart rate may be falsely detected due to body motion artifact or the like, and the accuracy is not sufficient if the heart rate information having the minimum value is simply employed as the minimum heart rate. Accordingly, in the embodiment, moving average processing is performed on the heart rate information acquired in a given measurement period and the minimum heart rate is obtained. This corresponds to the above described processing at S501.

At S402 in FIG. 28, the minimum heart rate obtained from the HRL histogram may be employed as the HRL minimum heart rate and the minimum heart rate obtained from the HRM histogram may be employed as the HRM minimum heart rate.

### 2.5 Specific Examples of Embodiments

In the above described embodiment, as shown in FIG. 2, the biological information processing system 100 may further include the body motion information acquisition unit 140 that acquires body motion information of the user. Further, the processing unit 115 calculates the first heart rate based on the resting heart rate obtained from the heart rate information having the signal level of the body motion information equal to or more than a given threshold value, and obtains the second heart rate from the heart rate information having the signal level of the body motion information less than the given threshold value. The processing unit 115 further performs comparison processing between the first heart rate and the second heart rate and determines the basal heart rate.

Here, the body motion information may be e.g., acceleration information (acceleration signal) acquired from an acceleration sensor, and the signal level of the body motion information equal to or more than the given threshold value is a condition that the acceleration level is equal to or more than the given Low level threshold value AccL (and equal to or less than AccH in the above described example). Specifically, the heart rate information having the signal level of the body motion information equal to or more than the given threshold value corresponds to the heart rate information determined as No at S502 and No at S504 in FIG. 29, and the HRM histogram is obtained as shown at S506 from the heart rate information. In the embodiment, the minimum heart rate obtained from the HRM histogram by the above described technique using FIG. 9 is employed as the resting heart rate, however, various modifications may be made to the technique of obtaining the resting heart rate from the heart rate information having the acceleration level equal to or more than AccL. Then, the first heart rate is calculated from the resting heart rate. Similarly, the heart rate information having the signal level of the body motion information less than the given threshold value corresponds to the heart rate information determined as Yes at S502 in FIG. 29, and the HRL histogram is obtained as shown at S503 from the heart rate information. Then, the minimum heart rate obtained from the HRL histogram is employed as the second heart rate.

Here, both the first and second heart rates are information conceptually corresponding to the basal heart rate. However, in the case where the measurement in the awake state is not performed or the like, it is impossible to obtain the resting heart rate and it is impossible to obtain the first heart rate. Or, in the case where the measurement in the sleep state is not performed, it is impossible to obtain the second heart rate. In this regard, in the technique of the embodiment, the first and second heart rates are treated as tentative information, and the appropriate basal heart rate is obtained by comparison between the heart rates. If at least one of the first and second heart rates has been appropriately obtained, it may be possible to make the basal heart rate obtained using the heart rates appropriate.

Note that the processing unit 115 may calculate the first heart rate by performing calculation processing using a given calculation formula, and specifically, like the formula (13), the first heart rate is calculated by dividing the resting heart rate by 1.2.

Further, the processing unit 115 employs the smaller one of the first heart rate and the second heart rate as the basal heart rate. Specifically, this corresponds to the processing at S405 to S407 in FIG. 28.

In this manner, it may be possible to obtain the basal heart rate from the first heart rate (HRM minimum heart rate/1.2) and the second heart rate (HRL minimum heart rate). The basal heart rate is the lowest heart rate that can be taken by the subject user, and the appropriate basal heart rate may be obtained by employing the smaller value.

Further, the processing unit 115 may calculate the basal heart rate based on the resting heart rate if the measurement period of the heart rate information is equal to or more than a predetermined period. Specifically, this corresponds to S401, etc. in FIG. 28.

In this manner, it may be possible to determine whether or not the processing with respect to the basal heart rate in response to the length of the measurement period is performed. If the measurement period is shorter, it is considered that sufficient data is not acquired and high-accuracy processing is difficult. In this case, the processing that may be lower in accuracy may be skipped without being forcibly executed, and processing load may be reduced. In the example in Ha. 28, the processing at S402 and the subsequent steps may be skipped.

Furthermore, the processing unit 115 may calculate calorie consumption of the user based on the resting heart rate.

Thereby, the calorie consumption may be obtained from the resting heart rate. Specifically, for example, the calculation using the formula (1), etc. may be performed. Note that, as described above, it may be possible to use the resting heart rate for other processing.

In addition, the above described embodiment may be applied to a biological information processing method (or an operation method of a biological information processing apparatus, an operation method of a biological information processing system) in which a heart rate information acquisition unit 110 acquires heart rate information of a user and a processing unit 115 obtains a resting heart rate as the heart rate information determined to correspond to a rest state of the user based on the heart rate information and calculates a basal heart rate based on the resting heart rate. Further, the heart rate information acquisition unit 110 may be applied to a biological information processing method (or an operation method of a biological information processing apparatus, an operation method of a biological information processing system) in which a heart rate information acquisition unit 110 acquires heart rate information of a user and a processing unit 115 obtains a basal heart rate as the heart rate information determined to correspond to a deep sleep state of the user based on the heart rate information and calculates a resting heart rate based on the obtained basal heart rate.

### 3. Third Embodiment

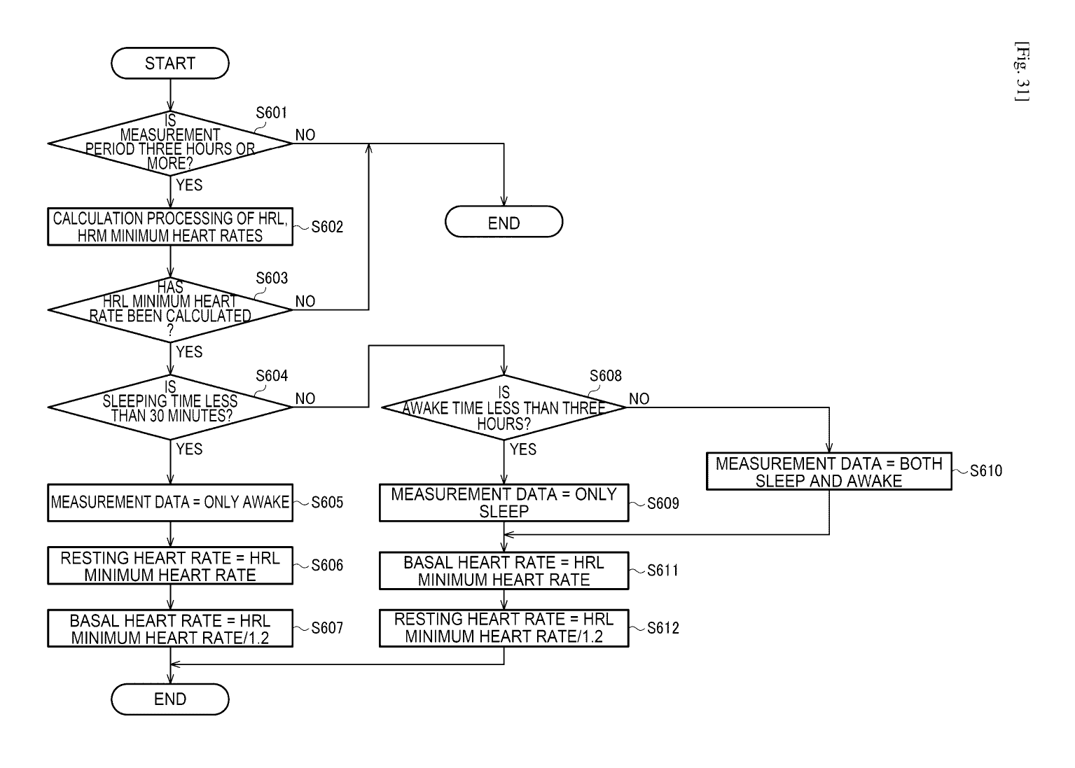

Next, the third embodiment will be explained. In the embodiment, when the basal heart rate, etc. are obtained, the sleep state is determined using body motion information. The system configuration example, the calculation technique of the HRL minimum heart rate, etc., and the processing using the basal heart rate, etc. are the same as those of the second embodiment, and their detailed explanation will be omitted. As below a specific flow of processing will be explained using a flowchart in FIG. 31, etc. Further, a modified example will be explained using FIG. 32, etc.

### 3.1 Flow of Processing

In the second embodiment, the processing with low load is realized by assuming that the HRL minimum heart rate is the basal heart rate and the HRM minimum heart rate is the resting heart rate. However, as described above, it is conceivable that the acceleration level is sufficiently lower even in the rest state. That is, in consideration that the HRL histogram used when the HRL minimum heart rate is obtained may contain both information in the sleep state and information in the rest state, it may be possible to accurately obtain the basal heart rate, etc.

Accordingly, in the embodiment, the sleep state is determined using body motion information, etc. and whether the heart rate information in the measurement period is data in only the awake state (or including the sleep state, but not sufficient) or data including the sleep state for the sufficient period is determined.

If the information is the data of only the awake state, the HRL histogram includes the data in the awake state with small body motion, and it is considered that the HRL minimum heart rate corresponds to the heart rate corresponding to the rest state, i.e., the resting heart rate. On the other hand, if the information is the data sufficiently including the sleep state, it is possible that the HRL histogram includes the information in the awake state (e.g., information in the rest state). However, generally, the heart rate in the awake state is higher than the heart rate in the sleep state, and it is considered that, consequently, the obtained HRL minimum heart rate corresponds to the heart rate corresponding to the sleep state, i.e., the basal heart rate.

The specific flow of the processing is shown in the flow chart of FIG. 31. When the processing is started, first, whether or not the measurement period is three or more hours is determined (S601), and, if No, the processing is ended and, if Yes, the HRL minimum heart rate is calculated (S602). Then, whether or not the HRL minimum heart rate has been calculated is determined (S603), and, if not, the processing is ended. Note that, as shown in FIG. 31, the HRM minimum heart rate may be obtained at S602.

If the HRL minimum heart rate has been calculated, whether or not the sleeping time in the measurement period is less than 30 minutes is determined (S604). The processing may be performed using an acceleration signal (or moving average thereof). As an example, whether or not the acceleration signal is Low level is determined in processing every four seconds, and, if the acceleration signal is Low level, the sleeping time may be obtained by increasing the accumulated value (increasing four seconds at intervals of four seconds).

If Yes at S604, there is no sleeping time or, if there is, its length is not sufficient, and the measurement data is determined to correspond to only the awake state (S605). In this case, it is considered that the HRL histogram contains no information in the sleep state, and the HRL minimum heart rate obtained at S602 is employed as the resting heart rate (S606). Then, the resting heart rate has been obtained, and thereby, the basal heart rate is calculated according to the formula (13) (S607).

On the other hand, if No at S604, whether or not the awake time is less than three hours is determined (S608). The awake time may be a time obtained by subtracting the sleeping time from the measurement period. If Yes at S608, there is no awake time or, if there is, its length is not sufficient, and the measurement data is determined to correspond to only the sleep state (S609). If No at S608, the measurement data is determined to correspond to both the sleep state and the awake state (S610). Note that the processing at S608 to S610 may be omitted except the embodiment using the determination result whether only the sleep state or both are contained (e.g., a modified example to be described later).

At either S609 or S610, the HRT histogram contains a sufficient amount volume of data in the sleep state. Accordingly, the HRL minimum heart rate obtained at S602 is employed as the basal heart rate (S611). Then, the basal heart rate has been obtained, and thereby, the resting heart rate is calculated according to the following formula (14) formed by transforming the formula (13) (S612).

resting heart rate=basal heart rate*1.2 (14)

In the above described embodiment, as shown in FIG. 2, the biological information processing system 100 further includes the body motion information acquisition unit 140 that acquires body motion information of the user. Further, the processing unit 115 determines whether the acquired heart rate information is information in the sleep state or information in the awake state based on the body motion information, and calculates the basal heart rate based on the determination result. Alternatively, the processing unit 115 determines whether the acquired heart rate information is information in the sleep state or information in the awake state based on the body motion information, and calculates the resting heart rate based on the determination result.

Here, the determination as to whether the acquired heart rate information is information in the sleep state or information in the awake state corresponds to the determination at S604, S608 in FIG. 31. That is, in the embodiment, acquisition of the information that the heart rate information in the period from t1 to t2 is in the sleep state of the heart rate information in the measurement period is not hindered, however, it is not necessary to acquire the detailed information to the extent. Specifically, it is only necessary to determine whether or not the heart rate information in the measurement period contains a sufficient amount of information of the sleep state (and contains a sufficient amount of information of the awake state).

Thereby, it is possible to perform processing of determining the sleep state. As described above, it is possible to perform the processing with respect to the basal heart rate from the heart rate information in the sleep state, and it is possible to perform the processing with respect to the resting heart rate from the heart rate information in the awake state. That is, the sleep state is determined in advance, and thereby, whether what can be directly obtained is the basal heart rate or the resting heart rate may be determined, and thereby, a determination that the information that can not be directly obtained should be estimated by calculation processing may be made.

Further, the processing unit 115 obtains the minimum heart rate obtained from the heart rate information determined to be information in the awake state as the resting heart rate, and calculates the basal heart rate based on the obtained resting heart rate. This corresponds to the processing at S606, S607 in FIG. 31.

Note that, in the flowchart of FIG. 31, even when the heart rate information is determined to contain the information in the awake state as at S610, the processing at S606, S607 is not performed. This is because, generally, a relationship of basal heart rate<resting heart rate holds, and the heart rate information contains the information in the sleep state at S610, when the minimum heart rate is obtained from the heart rate information, the minimum heart rate is the basal heart rate. That is, “the heart rate information determined to be information in the awake state” in the embodiment is, in the restrict sense, “the heart rate information sufficiently containing the information in the awake state and containing a predetermined or less amount of the information in the sleep state”. Note that the result is obtained because the sleep state, the awake state, or both of the states are determined with the measurement period as a unit in FIG. 31. In the case where it is possible to extract only the information corresponding to the awake state from the heart rate information in the measurement period, the extracted information may be naturally “the heart rate information determined to be the information in the awake state”.

In this manner, it may be possible to obtain the resting heart rate from the heart rate information in the awake state and calculate the basal heart rate from the obtained resting heart rate using the formula (13) or the like. That is, a determination that what is directly obtained is the resting heart rate and the basal heart rate is preferably calculated using the resting heart rate may be made, and it may be possible to make the obtained basal heart rate and resting heart rate appropriate.

On the other hand, the processing unit 115 may obtain the minimum heart rate obtained from the heart rate information determined to be information in the sleep state as the basal heart rate. Further, the unit may calculate the resting heart rate based on the obtained basal heart rate. This corresponds to the processing at S611, S612 in FIG. 31. That is, a determination that what is directly obtained is the basal heart rate and the resting heart rate is preferably calculated using the basal heart rate may be made.

### 3.2 Modified Example

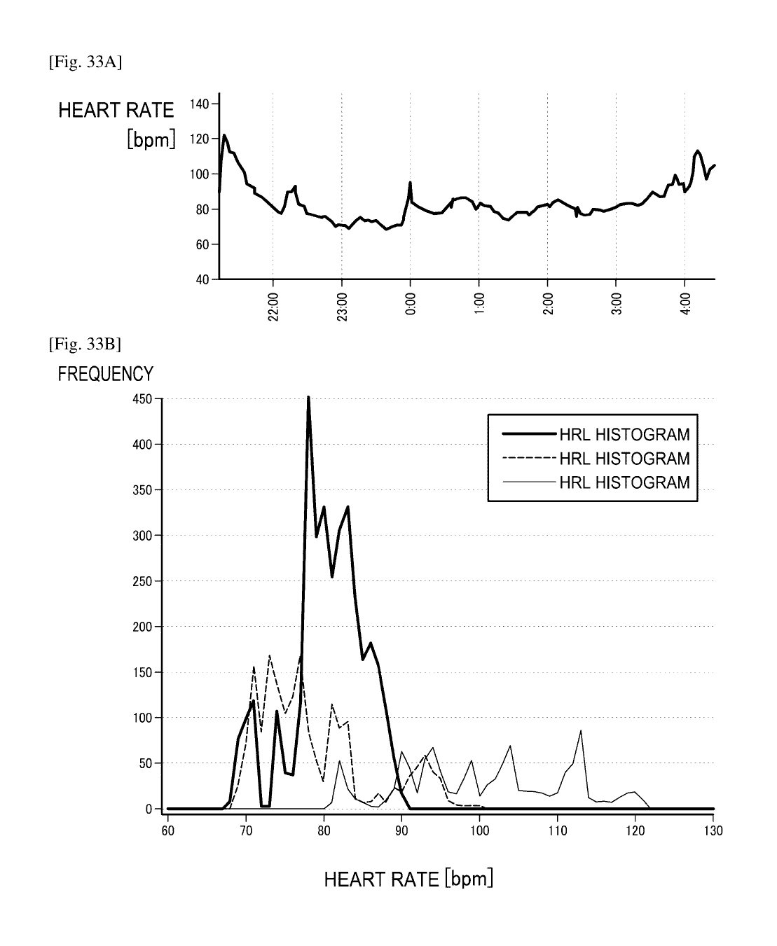

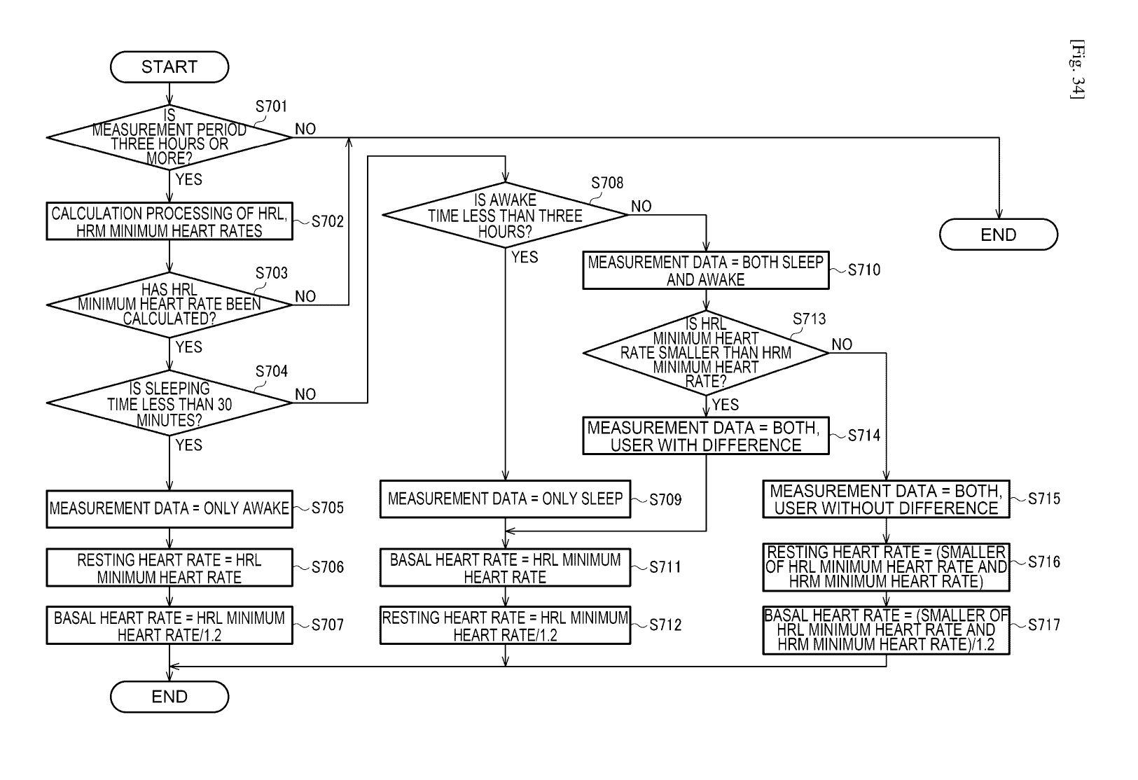

According to the above described techniques, the basal heart rate and the resting heart rate may be obtained regardless of the lifestyle or the like of the user. However, in the experiments by the applicant, it has been found that some users have values of heart rate information with smaller changes between the sleep state and the awake state than those of normal users. FIG. 32 shows actual measurement data of the user. FIG. 32 shows the data from 13:00 to 8:00 on the following day and contains both the data in the awake state and the data in the sleep state, in which the changes of the value of the heart rate information are smaller except the information from 6:00 when exercise was explicitly performed. Further, FIG. 33A, which will be described later, shows a graph representing changes of the heart rate information, and, in comparison to FIG. 30A as the graph of the normal user, it is clearly known that the changes of the heart rate information are smaller.

For example, in this case, it is considered that the heart rate in the sleep state is not sufficiently lower on the grounds that the user has a sleep apnea syndrome, drunk before sleep, or takes a drug that raises the heart rate. For the user, when the basal heart rate is set from the heart rate information in the sleep state, the heart rate information takes a value closer to the basal heart rate even in the awake state.

Accordingly, for example, the heart rate information HR satisfies the formula (11), and thereby, even when the user is actually awake, there is a risk that a false determination that the user is in the sleep state is made. Ha. 32 shows this point and the sleep state was determined in the hours in the awake state about from 16:30 to 17:00 or around 19:00.

Regarding the user, even when the measurement is performed in the sleep state, it is impossible to acquire the value to be originally set as the basal heart rate. Accordingly, in the modified example, with respect to the user having smaller differences in heart rate information between the sleep state and the awake state, the basal heart rate is calculated by special processing.

Specifically, the minimum heart rate obtained from the actually measured data is employed as the resting heart rate. When the resting heart rate is obtained, whether the object data is the data in the deep state or the data in the awake state is not considered. Further, the basal heart rate is obtained using the obtained resting heart rate and the formula (13). In this manner, the value expected to be lower to the extent in the deep sleep state for the subject user may be employed as the basal heart rate.

Note that the above described FIG. 30B shows the respective histograms of the normal user having the larger differences in heart rate information between the sleep state and the awake state, and, as clearly known from the drawing, the heart rate information tends to be larger as body motion is larger. Accordingly, as described above in the third embodiment, the HRL minimum heart rate may be used as the lowest heart rate as reference of the processing.

However, in the experiments of the applicant, it has found that the tendency does not necessarily apply to the user as in FIG. 32. FIG. 33A shows changes of the heart rate information of the user having smaller differences in heart rate information between the sleep state and the awake state, and FIG. 33B shows respective histograms of HRL, HRM, HRH obtained from the heart rate information. As known from FIG. 33B, for the user, HRM minimum heart rate<HRL minimum heart rate and the magnitude relationship of the body motion information and the magnitude relationship of the minimum heart rate are inversed. In this case, the information corresponding to the lowest heart rate is used as reference of the processing.

Therefore, in the modified example, both the HRL minimum heart rate and the HRM minimum heart rate are obtained in advance, and the smaller one is employed as the resting heart rate. The specific flow of processing is shown in the flowchart of FIG. 34.

S701 to S712 are the same as S601 to S612. Note that, at S702, as described above, the HRM minimum heart rate is obtained in addition to the HRL minimum heart rate. Further, to determine whether or not the differences in heart rate information between the sleep state and the awake state are smaller, it is necessary to compare the measurement data in both the sleep state and the awake state.

Accordingly, in the case where the data in the measurement period contains both the sleep state and the awake state, i.e., the determination at S710 is made in FIG. 34, the heart rate information is compared between the sleep state and the awake state. Here, like the second embodiment, comparison processing between the HRL minimum heart rate and the HRM minimum heart rate is performed according to the assumption that the HRL minimum heart rate corresponds to the heart rate in the sleep state and the HRM minimum heart rate corresponds to the heart rate in the awake state (S713).

Specifically, the processing at S713 may be realized by the determination as to whether (HRL minimum heart rate/HRM minimum heart rate)<ratio threshold value or (HRM minimum heart rate−HRL minimum heart rate)<difference threshold value.

If Yes at S713, a determination that the differences in heart rate information between the sleep state and the awake state are sufficiently large may be made (S714). In this case, it is not necessary to perform special processing, and the processing moves to S711 and the same processing as that of the above described third embodiment is performed. On the other hand, if No at S713, a determination that the differences in heart rate information between the sleep state and the awake state are smaller may be made (S715).

In this case, as described above, the minimum heart rate obtained from all measurement periods may be employed as the resting heart rate. Specifically, the smaller one of the HRL minimum heart rate and the HRM minimum heart rate is employed as the resting heart rate (S716), and the basal heart rate is calculated from the obtained resting heart rate and the formula (13) (S717).

By the processing of the modified example, the basal heart rate may be set to a lower value that should be originally taken. Note that, as described above, for the user, the value of the heart rate information in the sleep state is not lower to the obtained basal heart rate or a value Closer to that. As a result, in the case where the sleep determination using the basal heart rate set at S717 and the formula (11) is performed, it should be considered that there is a risk that the formula (11) is not satisfied though the user is actually sleeping, and a false determination of the awake state is made.

In the above described modified example, in the case where the awake heart rate information as the heart rate information determined to be the information in the awake state and the sleep heart rate information as the heart rate information determined to be the information in the sleep state are acquired and the difference or ratio between the awake heart rate information and the sleep heart rate information is equal to or less than a given threshold value, the processing unit 115 obtains the minimum heart rate in the measurement period as the resting heart rate.

Note that, in the above described modified example, in the case where the heart rate information in the measurement period contains information in both the sleep state and the awake state, the heart rate information at the middle acceleration level is employed as the awake heart rate information and the heart rate information at the low acceleration level is employed as the sleep heart rate information.

In this manner, as shown in FIGS. 32 and 33A, with respect to the user having the differences in heart rate information between the sleep state and the awake state, it may be possible to appropriately obtain the resting heart rate and appropriately calculate the basal heart rate.

As above, the three embodiments 1 to 3 and the modified example to which the invention is applied are explained, however, the invention is not limited to the respective embodiments 1 to 3 and the modified example as they are, and, on the implementation stage, the component elements may be modified and embodied without departing from the scope of the invention. Further, various inventions may be made by appropriately combining the plurality of component elements disclosed in the respective embodiments 1 to 3 and the modified example. For example, some component elements may be deleted from all component elements described in the respective embodiments 1 to 3 and the modified example. Furthermore, in the specification or the drawings, the terms described with different terms in broader senses or synonymous may be replaced by the different terms in any part of the specification or the drawings. Thus, various modifications or applications may be made without departing from the scope of the invention.

1. A biological information processing system comprising:
a heart rate information acquisition unit that acquires heart rate information of a user;
a determination unit that determines a basal heart rate based on the heart rate information; and
an update unit that determines an update condition of the basal heart rate and performs update processing of the basal heart rate if a determination that the update condition is satisfied is made.

2. The biological information processing system according to claim 1, wherein the update unit increases the basal heart rate if the update condition is not satisfied in a predetermined period or at a predetermined number of times.

3. The biological information processing system according to claim 2, wherein the update unit counts a number of times of repetition from a start of measurement to an end of the measurement of the heart rate information as the predetermined number of times.

4. The biological information processing system according to claim 3, further comprising a body motion information acquisition unit that acquires body motion information from a body motion sensor,
wherein the update unit counts a number of times when body motion of the user is determined to be smaller of the number of times of repetition based on the body motion information in a period from the start of the measurement to the end of the measurement as the predetermined number of times.

5. The biological information processing system according to claim 1, wherein the update unit performs determination of the update condition of the basal heart rate based on a minimum heart rate obtained from the heart rate information.

6. The biological information processing system according to claim 5, wherein the update unit obtains the minimum heart rate by performing moving average processing on the heart rate information acquired in a given minimum heart rate measurement period.

7. The biological information processing system according to claim 6, wherein the update unit obtains a histogram representing a relationship between heart rate values and frequencies at which the respective heart rate values are detected based on the heart rate information acquired in the minimum heart rate measurement period, and, in a range of the heart rate value from x to x+n (x, n are given positive numbers), obtains x for which the frequency exceeds a given frequency threshold value and the value is minimum as the minimum heart rate.

8. The biological information processing system according to claim 1, wherein the determination unit performs processing of replacing a default value of the basal heart rate set by the biological information processing system by the minimum heart rate obtained from the heart rate information, and
the update unit performs the update processing of updating the basal heart rate determined by the determination unit.

9. The biological information processing system according to claim 8, further comprising a body motion information acquisition unit that acquires body motion information from a body motion sensor,
wherein, if a determination that a period in which body motion of the user is determined to be smaller is contained in a given minimum heart rate measurement period is made based on the body motion information in the minimum heart rate measurement period, the determination unit employs the minimum heart rate obtained based on the heart rate information in the minimum heart rate measurement period as the basal heart rate.

10. The biological information processing system according to claim 1, further comprising a body motion information acquisition unit that acquires body motion information from a body motion sensor,
wherein the update unit determines the update condition of the basal heart rate based on the body motion information.

11. The biological information processing system according to claim 2, wherein the predetermined number of times is a number of times when a determination of the update condition of the basal heart rate is made.

12. The biological information processing system according to claim 1, wherein the update unit increases the basal heart rate if a predetermined period lapses.

13. The biological information processing system according to claim 1, further comprising a health degree information calculation unit that obtains relative information between the basal heart rate and the heart rate information, and obtains health degree information representing a health degree based on the relative information.

14. A biological information processing system comprising:
a heart rate information acquisition unit that acquires heart rate information of a user; and
a processing unit that obtains a resting heart rate as the heart rate information determined to correspond to a rest state of the user based on the heart rate information, and calculates a basal heart rate based on the resting heart rate.

15. The biological information processing system according to claim 14, further comprising a body motion information acquisition unit that acquires body motion information of the user,
wherein the processing unit calculates a first heart rate based on the resting heart rate obtained from the heart rate information having the body motion information at a signal level equal to or more than a given threshold value, a second heart rate from the heart rate information having the body motion information at the signal level less than the given threshold value, and determines the basal heart rate by comparison processing between the first heart rate and the second heart rate.

16. The biological information processing system according to claim 15, wherein the processing unit employs smaller one of the first heart rate and the second heart rate as the basal heart rate.

17. The biological information processing system according to claim 15, wherein the processing unit calculates the first heart rate by performing calculation processing on the resting heart rate using a given calculation formula.

18. The biological information processing system according to claim 14, further comprising a body motion information acquisition unit that acquires body motion information of the user,
wherein the processing unit determines whether the acquired heart rate information is information in a sleep state or information in an awake state based on the body motion information, and calculates the basal heart rate based on a determination result.

19. The biological information processing system according to claim 18, wherein the processing unit obtains a minimum heart rate obtained from the heart rate information determined to be the information in the awake state as the resting heart rate, and calculates the basal heart rate based on the obtained resting heart rate.

20. The biological information processing system according to claim 18, wherein, in the case where awake heart rate information as the heart rate information determined to be the information in the awake state and sleep heart rate information as the heart rate information determined to be the information in the sleep state are acquired and a difference or ratio between the awake heart rate information and the sleep heart rate information is equal to or less than a given threshold value, the processing unit obtains a minimum heart rate in a measurement period as the resting heart rate.

21. The biological information processing system according to claim 18, wherein, in the case where the heart rate information is determined to be information in the sleep state, the processing unit employs a minimum heart rate obtained from the heart rate information as the basal heart rate.

22. The biological information processing system according to claim 18, wherein the processing unit calculates the basal heart rate by performing calculation processing on the resting heart rate using a given calculation formula.

23. The biological information processing system according to claim 14, wherein, in the case where a measurement period of the heart rate information is equal to or more than a predetermined period, the processing unit calculates the basal heart rate based on the resting heart rate.

24. A biological information processing system comprising:
a heart rate information acquisition unit that acquires heart rate information of a user; and
a processing unit that obtains a basal heart rate as the heart rate information determined to correspond to a deep sleep state of the user based on the heart rate information, and obtains a resting heart rate based on the obtained basal heart rate.

25. The biological information processing system according to claim 24, wherein the processing unit calculates calorie consumption of the user based on the resting heart rate.

26. The biological information processing system according to claim 24, further comprising a body motion information acquisition unit that acquires body motion information of the user,
wherein the processing unit determines whether the acquired heart rate information is information in a sleep state or information in an awake state based on the body motion information, and calculates the resting heart rate based on a determination result.

27. The biological information processing system according to claim 24, wherein the processing unit calculates the resting heart rate by performing calculation processing on the basal heart rate using a given calculation formula.

28. An electronic apparatus comprising the biological information processing system according to claim 1.

29. A server system comprising the biological information processing system according to claim 1.

30. A biological information processing method comprising:
acquiring heart rate information of a user;
determining a basal heart rate based on the heart rate information; and
determining an update condition of the basal heart rate and performing update processing of the basal heart rate if a determination that the update condition is satisfied is made.

31. A biological information processing method comprising:
acquiring heart rate information of a user; and
obtaining a resting heart rate as the heart rate information determined to correspond to a rest state of the user based on the heart rate information, and calculating a basal heart rate based on the resting heart rate.

32. A biological information processing method comprising:
acquiring heart rate information of a user; and
obtaining a basal heart rate as the heart rate information determined to correspond to a deep sleep state of the user based on the heart rate information, and calculating a resting heart rate based on the obtained basal heart rate.
